SEQUENCE LISTING (2 sequences)

SEQ ID NO: 1 - DNA, 23 bp, Artificial Sequence
  Description of Artificial Sequence Synthetic primer
agacctacactgttggctgtgag

SEQ ID NO: 2 - DNA, 21 bp, Artificial Sequence
  Description of Artificial Sequence Synthetic primer
gactggaagcccttttcagag

(19) United States
(12) Patent Application Publication
(10) Pub. No.: US 2019/0234937 A1
(43) Pub. Date: Aug. 1, 2019

(54) BIOMATRIX SCAFFOLDS FOR USE IN DIAGNOSING AND MODELING CANCER

(71) Applicant: The University of North Carolina at Chapel Hill, Chapel Hill, NC (US)

(72) Inventors: Andrew Zhuang Wang, Durham, NC (US); Xi Tian, Chapel Hill, NC (US); Lola M. Reid, Chapel Hill, NC (US)

(73) Assignee: The University of North Carolina at Chapel Hill, Chapel Hill, NC (US)

(21) Appl. No.: 16/317,323

(22) PCT Filed: Jul. 11, 2017
(86) PCT No.: PCT/US17/41500

§ 371 Date: Jan. 11, 2019

Related U.S. Application Data
(60) Provisional application No. 62/361,306, filed on Jul. 12, 2016.
(60) Provisional application No. 62/460,056, filed on Feb. 16, 2017.

(51) Int. Cl. G01N 33/50; G01N 33/574; C12N 5/09; C12N 5/00

(52) U.S. Cl. CPC G01N 33/5011; G01N 33/574; A61K 45/06; C12N 5/0068; G01N 2500/10; C12N 5/0693

(57) ABSTRACT

Described herein are in vitro cancer metastasis models that recapitulate biological facets of organ specificity as indicated by the behavior of human colorectal cancer cells when on substrata of liver and lung biomatrix scaffolds, organ-specific matrix extracts generated by novel decellularization strategies. Tumor cells spontaneously formed colonies that mimicked in vivo metastatic lesions in terms of histology, biology and genetic and other phenotypic traits. Not to be bound by theory, it is postulated that responses of tumors to radiation or chemotherapeutic agents proved dependent on the matrix substratum used; thus, thus the scaffolds are believed to provide a powerful tool for cancer research and diagnosis.

### CROSS-REFERENCE TO RELATED APPLICATIONS

This application claims priority under 35 U.S.C. 119(e) to U.S. Ser. No. 62/361,306, filed Jul. 12, 2016, and U.S. Ser. No. 62/460,056, filed Feb. 16, 2017, the entirety of which are incorporated by reference herein.

### BACKGROUND

Metastasis is the primary cause of morbidity and mortality in cancer. A major challenge in cancer research has been the lack of ex vivo or in vitro models that recapitulate the biology of cancer metastasis, especially with respect to organ specificity, a key characteristic of metastasis. Thus, better tools for cancer research and therapeutics development require better models of metastatic growth.

Organ-specificity is an important hallmark of cancer metastasis as tumor cells are known to have predilection to metastasize to certain organs. Furthermore, the treatment responses of metastatic lesions can be dependent on the organ in which the metastatic lesions occur and even if within the same patient. However, existing in vitro/ex vivo model systems do not reflect such organ specificity, likely due to the lack of variables in the organ-microenvironment in these tumor models. Although in vivo genetically engineered mouse models do have organ specificity, they are difficult to study and very costly.

Clinically, a challenge has been the lack of tools to predict which tumor will metastasize and to which tissues or organs it will metastasize. An assay that can predict sites of metastasis will be of immense clinical value and can alter cancer management.

### SUMMARY

Aspects of this invention relate to methods, kits, and compositions related to diagnosing and/or characterizing a cancer or tumor. In any of the embodiments described relating to the cancer or tumor herein, the cancer or tumor may be malignant. In certain embodiments, the cancer or tumor may be characterized by its potential to metastasize to specific tissues and/or the responsiveness of one or more of such metastases to respond to one or more cancer therapies and/or chemotherapeutic agents described herein below.

Method embodiments disclosed herein comprise seeding one or more biomatrix scaffolds with cells from a tumor or cancer.

In some of these method embodiments, the cells may be from (1) cells of a tumor biopsy or sample taken from the patient diagnosed with a cancer or tumor or (2) cells from a cell line of the same cancer or tumor type as that of the patient. In some of these method embodiments, the cells are analyzed for their ability to form a colony of growing cells. In some embodiments, the colony is on the substratum of the biomatrix scaffold. In some of these method embodiments, the cells form a colony. In some of these method embodiments, the cells are analyzed for their ability to become 3-dimensional colonies. In some of these method embodiments, the cells form a 3-dimensional colony. Not to be bound by theory, it is believed that such 3-dimensional colony formation may be influenced by the chemistry of the substratum of the respective biomatrix scaffolds. In some of these method embodiments, the cells are analyzed for their ability to express genes or to secrete proteins or other factors. Non-limiting examples of these genes include: pluripotency genes (e.g. OCT4, SOX2, KLF4, KLF5, SALL4, NANOG, BMi-1), stem cell genes (e.g. EpCAM, LGR5/LGR6, CXCR4, one or more of the many variants of the CD44 family of genes encoding hyaluronan receptors, multidrug resistance genes (e.g. mdr gene family, p-glycoproteins), genes encoding enzymes that dissolve extracellular matrix components (e.g. hyaluronidase, collagenases, elastase, matrix-degrading metalloproteinases); non-limiting examples of these proteins include: the proteins encoded by the genes noted above (e.g. CD44, p-glycoproteins, the matrix degrading enzymes); non-limiting examples of other factors relevant to this analysis include those associated with exosome production, microRNAs, and qualitative or quantitative independence of paracrine signaling from mesenchymal feeders. In some embodiments of these method embodiments, this analysis may occur once the cells have been seeded and attached onto one or more biomatrix scaffolds. In some embodiments of these method embodiments, the cells may attach to the substratum of tissue-specific forms of the one or more biomatrix scaffolds.

In some of these method embodiments, the one or more biomatrix scaffolds originating from one or more predetermined organs, respectively, of a human body. In further embodiments, the predetermined organs are selected based on the cancer or tumor type of the patient—e.g. to include organs known to be associated with metastases of the cancer or tumor type or to include the organ in which the cancer or tumor was found. In all method embodiments, each biomatrix scaffold recapitulates the biological facets of the tissue from which it originated.

The seeding method may optionally be used to determine a potential of a tumor to metastasize within a patient diagnosed with a cancer or tumor. For example, metastasis into an organ may be predicted if the tumor or cancer cell forms a colony of growing cells on a biomatrix scaffold. The predicted metastases may be further determined to be localized to the organ from which said scaffold was derived. Thus, in some embodiments, metastasis into one or more predetermined organs is predicted in vivo if the cells form a colony of growing cells on the one or more biomatrix scaffolds, respectively, originating therefrom.

The seeding method may also be used to determine the appropriate treatment for a tumor in a patient diagnosed with a cancer or tumor. This may be done in addition to determining a potential of a tumor to metastasize or independently therefrom.

Any of the above disclosed method embodiments may further comprise characterizing the colony based on its histology, exosome production, microRNA production, interactions with mesenchymal cells, and/or gene expression profile.

Any of the above disclosed method embodiments may further comprise screening the colony for responsiveness to one or more cancer therapies and/or chemotherapeutic agents or treating the colony with one or more cancer therapies and/or chemotherapeutic agents. Such cancer therapies and/or chemotherapeutic agents include, but are not limited to, one or more doses of radiation therapy, immunotherapy, endocrine therapy, molecular therapy, and/or one or more type of chemotherapy such as an anthracycline, an alkylating agent, a platinum-based agent, an anti-metabolite, a topoisomerase inhibitor, or a mitotic inhibitor. Such cancer therapies and/or chemotherapeutic agents may optionally be selected based on the cancer or tumor type of the patient. In certain embodiments, the responsiveness of the cancer or tumor to the one or more therapies and/or agents in vivo in an organ may be correlated with the responsiveness of the cancer or tumor cells to the one or more therapies or agents when seeded on a biomatrix scaffold prepared from the organ.

Aspects of the disclosure also relate to kits comprising one or more biomatrix scaffolds originating from one or more predetermined organs, respectively, of a human body and instructions to carry out one or more of the methods disclosed herein above. Further embodiments contemplate kits further comprising one or more cancer therapies and/or chemotherapeutic agents; tools or instructions for obtaining cells of a tumor biopsy or sample(s) taken from the patient diagnosed with a cancer or tumor; cells from a cell line from a particular cancer or tumor type; and/or any reagents, media, or other components required to carry out the above disclosed methods.

Further aspects of the disclosure contemplate an artificial tumor or metastases model specific to one or more predetermined organs of a human body generated by the seeding method disclosed herein above.

### BRIEF DESCRIPTION OF THE DRAWINGS

FIG. 1 shows that the Biomatrix Scaffolds (BMSs) recapitulate tissue-specific microenvironments found in vivo. (a) Schematic depicting methodology used for studying metastatic disease in vitro. (b) Scanning electron micrographs of lung and liver BMSs. Scale bars, 100 nm. (c) Analysis of growth factors and cytokines present in lung tissues before and after decellularization (n=4). Differences in the relative abundance of signaling molecules were determined using paired t-tests. Data represent mean±S.E.M.; * p<0.001; (d) Heat map comparing the composition and relative abundance of proteins present in liver and lung BMSs (n=4). Hierarchical clustering was performed using z-score normalized label-free quantification intensities.

FIG. 2 demonstrates that colorectal cancer cells spontaneously form 3D engineered metastases when cultured on liver and lung BMSs. (a) Scanning electron micrographs of HT-29 (left), SW480 (middle), and Caco2 (right) cells grown on plastic, collagen, Matrigel, liver BMSs, and lung BMSs. Scale bars, 50 μm. (b) Seeding efficiencies and (c) growth rates of HT-29 (left), SW480 (middle), and Caco2 (right) cells seeded on plastic, collagen, Matrigel, liver BMSs, and lung BMSs (n=3). Data represent mean±S.E.M. Differences in seeding efficiency and growth rate were determined using a one-way ANOVA with Tukey's multiple comparison post-test. Statistical significance is indicated with letters above (P<0.05). Groups that share the same letter are not significantly different.

FIG. 3 shows that engineered liver metastases are comparable to liver metastases found in vivo. (a) Engineered HT-29 liver metastases (left panel), liver metastases formed following intrasplenic injection of HT-29 cells (middle panel), and liver metastases biopsied from late stage human colorectal cancer patients (right panel) all demonstrate classic histologic features of liver metastases of gastrointestinal origin. Scale bars, 20 μm. (b) Hierarchal cluster analysis of average global gene expression patterns displayed by HT-29 cells grown on plastic (“Plastic”), Matrigel (“Matrigel”), liver BMSs (“Engineered liver metastases”), and in vivo liver metastases derived from intrasplenic injection of HT-29 cells (“In vivo liver metastases”) (n=4).

FIG. 4 demonstrates that engineered metastases demonstrated increased metastatic potential in vivo. (a,c) Bioluminescence images of animals 30 days post (a) direct hepatic injection or (c) tail vein injection with HT-29-luc2 cells isolated from plastic, collagen, Matrigel, liver BMSs, and lung BMSs. (b,d) Table summarizing the number of animals that developed liver and lung metastases post (b) direct hepatic injection or (d) tail vein injection of HT-29-luc2 cells.

FIG. 5 shows that CRC cells grown on different substrata's respond differently to chemotherapeutics and radiotherapy. (a) Responses of CRC cells grown on plastic, collagen, Matrigel, liver BMSs, and lung BMSs to chemotherapeutics (n=6). Data represent mean±S.E.M. Differences in therapeutic responses were determined using a one-way ANOVA with Tukey's multiple comparison post-test. Statistical significance is indicated with letters above (P<0.05). Groups that share the same letter are not significantly different. (b) Responses of CRC cells grown on plastic, collagen, Matrigel, liver BMSs, and lung BMSs to radiotherapy (n=3). Data represent mean±S.E.M.

FIG. 6 demonstrates that decellularization of lung and liver tissues produces BMSs containing tissue specific signaling molecules (a) Macroscopic and H&E images of lung (left) and liver (right) before and after decellularization. Scale bars, 130 μm. (b) Assessment of DNA content present in lungs and livers before and after decellularization (n=3). Data represent mean±S.E.M. Differences in DNA content were determined using t-test. (****P<0.0001). (c) Analyses of growth factors and cytokines present in lung and liver BMSs (n=4). Mean pixel intensity values for signaling molecules present in liver BMSs were obtained from a previous study.

FIG. 7 shows that the composition of extracellular matrix components differs between liver and lung BMSs. Heat map comparing the relative abundance of collagens, elastins, fibronectins, and laminins that are differentially expressed in liver and lung BMSs (n=4). Hierarchical clustering was performed using z-score normalized label-free quantification intensities.

FIG. 8 depicts transmission electron micrographs of the engineered liver metastases depicting (a) the interface between the colony and the biomatrix scaffold, (b) tight junctions between cells, and (c) areas of necrosis. (a-b) Scale bars, 2 μm. (c) Scale bar, 10 μm. Biomatrix scaffold in the left panel is indicated by “B”. A tight junction in the middle panel is indicated by a dotted box. A necrotic area in the right panel is indicated by “N”.

FIG. 9 shows that engineered metastases grow relatively slowly due to reduced proliferation rates. (a) Quantification of cells undergoing apoptosis, Cleaved Caspase 3 positive, in CRC cultures grown on plastic, collagen, Matrigel, liver BMSs, and lung BMSs using flow cytometry (n=3). (b) Quantification of the number of cells cultured on different substrata that underwent S-phase, EdU positive, over a four hour labeling period as assessed by flow cytometry (n=3). Data represent mean±S.E.M. Differences in apoptosis and proliferation rate were assessed using a one-way ANOVA with Tukey's multiple comparison post-test. Statistical significance is indicated with letters above (P<0.05) using. Groups that share the same letter are not significantly different.

FIG. 10 depict representative images of HT-29 (left), SW480 (middle), and Caco2 (right) cells grown on plastic, collagen, Matrigel, liver BMSs and lung BMSs. Scale bars, 20 μm.

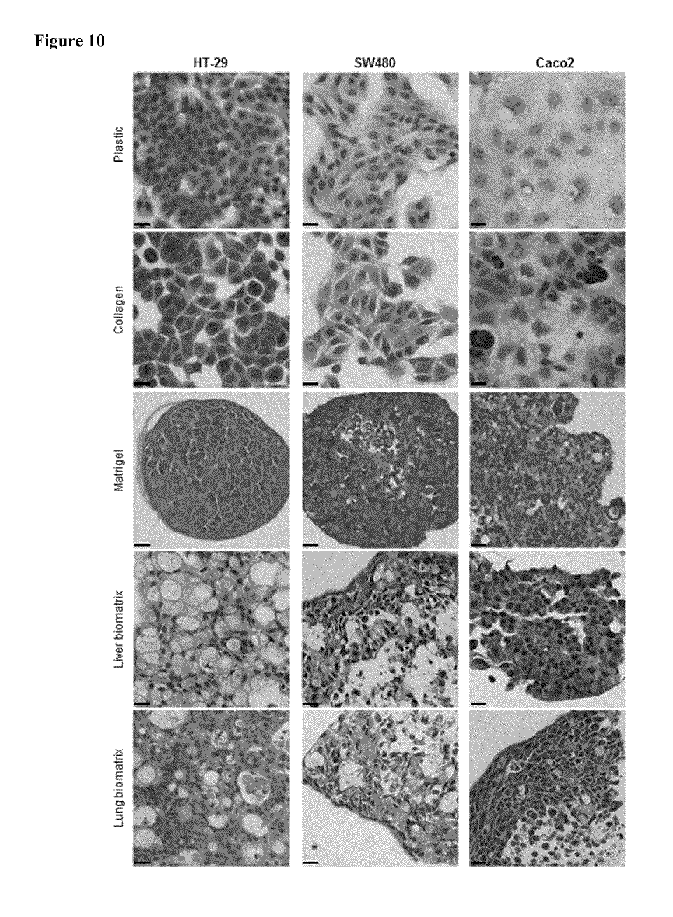

FIG. 11 demonstrates that engineered liver metastases and in vivo metastases demonstrate comparable gene signatures (a) Global gene expression profiles of In vivo HT-29 liver metastases and (b) engineered liver metastases were assessed by microarray analysis (n=4). Liver metastases in (a) is indicated by white arrow. Scale bars, 100 μm. (c) Venn diagram depicting the number of genes up-regulated in Matrigel cultures, engineered metastases, and in vivo liver metastases when compared to cells grown on plastic. Gene ontology analysis reveals that many commonly up-regulated genes are associated with angiogenesis, cellular adhesion, and drug metabolism (d) Timp1 gene expression in HT-29, SW480, and Caco2 cells grown on plastic, collagen, Matrigel, liver and lung BMSs as assessed by real-time qPCR (n=4). Data represent mean±S.E.M. Differences in Timp1 expression were determined using a one-way ANOVA with Tukey's multiple comparison post-test. Statistical significance is indicated with letters above (P<0.05). Groups that share the same letter are not significantly different.

FIG. 12 depicts whole organ ex vivo bioluminescent imaging and histological analyses were used to confirm the presence of metastatic lesions identified based on whole animal bioluminescent images following tail vein injection (a) or direct hepatic injection (b) of HT29-luc2 cells. Representative liver and lung metastases identified by histology are indicated by an arrow. Scale bars, 20 μm.

FIG. 13 shows that hypoxic preconditioning does not increase the metastatic potential of HT-29 cells grown on plastic. (a) Bioluminescence images of animals 30 days post tail vein injection of HT-29-luc2 cells cultured under normal and hypoxic conditions. (b) Table summarizing the number of animals that developed lung metastases.

FIG. 14 depicts characterization of the relative susceptibility to anoikis and invasive potential displayed by CRC cells grown on different culture substrata. (a) Relative resistance of HT-29, SW480, and Caco2 cells grown on plastic, collagen, Matrigel, liver BMSs, and lung BMSs to undergoing anoikis following culture on hydrogel-coated plates (n=3). (b) Invasion potential of CRC cells grown on different substrata as determined using a transwell invasion assay (n=3). Data represent mean±S.E.M. Differences in survival and invasion were determined using a one way ANOVA with Tukey's multiple comparison post-test. Statistical significance is indicated with letters above (P<0.05). Groups that share the same letter are not significantly different.

### DETAILED DESCRIPTION

Embodiments according to the present disclosure will be described more fully hereinafter. Aspects of the disclosure may, however, be embodied in different forms and should not be construed as limited to the embodiments set forth herein. Rather, these embodiments are provided so that this disclosure will be thorough and complete, and will fully convey the scope of the invention to those skilled in the art. The terminology used in the description herein is for the purpose of describing particular embodiments only and is not intended to be limiting. All references mentioned herein and throughout the application are incorporated by reference.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. While not explicitly defined below, such terms should be interpreted according to their common meaning.

The terminology used in the description herein is for the purpose of describing particular embodiments only and is not intended to be limiting of the invention. All publications, patent applications, patents and other references mentioned herein are incorporated by reference in their entirety.

The practice of the present technology will employ, unless otherwise indicated, conventional techniques of tissue culture, immunology, molecular biology, microbiology, cell biology, and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al. eds (1996) Weir's Handbook of Experimental Immunology.

Unless the context indicates otherwise, it is specifically intended that the various features of the invention described herein can be used in any combination. Moreover, the disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

### Definitions

As used in the description of the invention and the appended claims, the singular forms “a,” “an” and “the” are intended to include the plural forms as well, unless the context clearly indicates otherwise.

The term “about,” as used herein when referring to a measurable value such as an amount or concentration (e.g., the percentage of collagen in the total proteins in the biomatrix scaffold) and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”).

The terms “buffer” and/or “rinse media” are used herein to refer to the reagents used in the preparation of the biomatrix scaffolds.

As used herein, the term “cell” refers to a eukaryotic cell. In some embodiments, this cell is of animal origin and can be a stem cell or a somatic cell. The term “population of cells” refers to a group of one or more cells of the same or different cell type with the same or different origin. In some embodiments, this population of cells may be derived from a cell line; in some embodiments, this population of cells may be derived from a sample of an organ or tissue.

As used herein, the term “comprising” is intended to mean that the compositions and methods include the recited elements, but do not exclude others. As used herein, the transitional phrase “consisting essentially of” (and grammatical variants) is to be interpreted as encompassing the recited materials or steps “and those that do not materially affect the basic and novel characteristic(s)” of the recited embodiment. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.” “Consisting of” shall mean excluding more than trace elements of other ingredients and substantial method steps for administering the compositions disclosed herein. Aspects defined by each of these transition terms are within the scope of the present disclosure.

The term “culture” or “cell culture” means the maintenance of cells in an artificial, in vitro or ex vivo two dimensional (2D, monolayer) or three dimensional (3D) environment (polarized shapes of cells when on certain forms of matrix or when floating), in some embodiments as adherent cells (e.g. monolayer cultures) or as floating aggregates cultures of spheroids or organoids. The term “spheroid” indicates a floating aggregate of cells all being the same cell type (e.g. an aggregate from a cell line); an “organoid” is a floating aggregate of cells comprised of multiple cell types. In some embodiments, the organoid may be an aggregate of epithelia and one or more mesenchymal cell types comprising endothelia and/or stromal or stellate cells. A “cell culture system” is used herein to refer to culture conditions in which a population of cells may survive or be grown.

“Culture medium” is used herein to refer to a nutrient solution for the culturing, growth, or proliferation of cells. In some embodiments, it comprises one or more of amino acids, vitamins, salts, lipids, minerals, trace elements) and mimicking the chemical constituents of interstitial fluid. Culture medium may be characterized by functional properties such as, but not limited to, the ability to maintain cells in a particular state (e.g. a pluripotent state, a quiescent state, etc.), to mature cells—in some instances, specifically, to promote the differentiation of stem/progenitor cells into cells of a particular lineage. A non-limiting example of culture medium used for stem/progenitors is Kubota's Medium, which is further defined herein below. In some embodiments the medium may be a “seeding medium” used to present or introduce cells into a given environment.

More specifically, a “basal medium” is a buffer comprised of amino acids, sugars, lipids, vitamins, minerals, salts, trace elements and various nutrients in compositions that mimic the chemical constituents of interstitial fluid around cells. Such media may optionally be supplemented with serum to provide requisite signaling molecules (hormones, growth factors) needed to drive a biological process (e.g. proliferation, differentiation) or as a source of inhibitors to enzymes used typically in the preparation of cell suspensions. Although the serum can be autologous to the cell types used in cultures, it is most commonly serum from animals routinely slaughtered for agricultural or food purposes such as serum from cows, sheep, goats, horses, etc. Media supplemented with serum may be optionally referred to as serum supplemented media (SSM).

As used herein, “differentiation” means that specific conditions cause cells to mature to adult cell types that produce adult specific gene products.

The terms “equivalent” or “biological equivalent” are used interchangeably when referring to a particular molecule, biological, or cellular material and intend those having minimal homology while still maintaining desired structure or functionality.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample; further, the expression level of multiple genes can be determined to establish an expression profile for a particular sample.

As used herein, the term “functional” may be used to modify any molecule, biological, or cellular material to intend that it accomplishes a particular, specified effect.

The term “gene” as used herein is meant broadly to include any nucleic acid sequence transcribed into an RNA molecule, whether the RNA is coding (e.g., mRNA) or non-coding (e.g., ncRNA).

As used herein, the term “generate” and its equivalents (e.g. generating, generated, etc.) are used interchangeably with “produce” and its equivalents when referring to the method steps that yield a particular model colony, organ, or organoid.

The term “isolated” as used herein refers to molecules or biologicals or cellular materials being substantially free from other materials.

“Kubota's Medium” as used herein refers to a serum-free, wholly defined medium designed for endodermal stem cells and enabling them to expand clonogenically in a self-replicative mode of division (especially if on hyaluronan substrata or in 3D hyaluronan hydrogels). Kubota's medium may refer to any basal medium containing no copper, low calcium (<0.5 mM), insulin, transferrin/Fe, a mix of purified free fatty acids bound to purified albumin and, optionally, also high density lipoprotein. Kubota's Medium or its equivalent is used serum-free, especially in culture selection for endodermal stem cells, and contains only a defined mix of purified signals (insulin, transferrin/Fe), lipids, and nutrients. In some embodiments, it can be used transiently as a SSM using low (typically 5% or less) levels of serum for the seeding process of introducing cells into the matrix scaffolds and in order to inactivate enzymes used in preparing cell suspensions; switching to the serum-free Kubota's Medium as quickly as possible (e.g. within 5-6 hours) is optimal.

In certain embodiments, the medium is comprised of a serum-free basal medium (e.g., RPMI 1640 or DME/F12) containing no copper, low calcium (<0.5 mM) and supplemented with insulin (5 μg/mL), transferrin/Fe (5 μg/mL), high density lipoprotein (10 μg/mL), selenium (10⁻¹⁰ M), zinc (10⁻¹² M), nicotinamide (5 μg/mL), and a mixture of purified free fatty acids bound to a form of purified albumin. Non-limiting, exemplary methods for the preparation of this media have been published elsewhere, e.g., Kubota H, Reid L M, Proceedings of the National Academy of Sciences (USA) 2000; 97:12132-12137, Y. Wang, H. L. Yao, C. B. Cui et al. Hepatology. 2010; 52(4):1443-54, Turner et al; Journal of Biomedical Biomaterials. 2000; 82(1): pp. 156-168; Y. Wang, H. L. Yao, C. B. Cui et al. Hepatology. 2010 October 52(4):1443-54, the disclosures of which is incorporated herein by reference. Variants of Kubota's Medium can be used for certain cell types by providing additional factors and supplements to allow for expansion under serum free conditions. For example, Kubota's Medium may be modified to enable transit amplifying cells or committed progenitors (e.g. hepatoblasts) and other maturational lineage stages later than stem cell populations to survive and expand ex vivo under serum-free conditions. One example of this is Kubota's Medium modified for ex vivo expansion of hepatoblasts and their descendants, committed progenitors: serum-free Kubota's Medium is further supplemented with hepatocyte growth factor (HGF), epidermal growth factor (EGF), basic fibroblast growth factor (bFGF), and sometimes vascular endothelial growth factor (VEGF). The resulting cell expansion occurs with minimal (if any) self-replication. The medium is especially effective if the cells are on substrata of type IV collagen and laminin or embedded in 3-D hydrogels containing more than 50% type IV collagen and laminin.

The terms “nucleic acid,” “polynucleotide,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers.

A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

As used herein, the term “organ” refers to a structure which is a specific portion of an individual organism, where a certain function of the individual organism is locally performed and which is morphologically independent. Non-limiting examples of organs include the skin, blood vessels, cornea, kidney, heart, liver, umbilical cord, intestine, nerve, lung, placenta, pancreas, and brain. Organs may be used as a tissue source; for example, fetal, neonatal, pediatric (child), or adult organs may be used to derive cell populations of interest for uses disclosed herein. The terms “tissue” is used herein to refer to tissue of a living or deceased organism or any tissue derived from or designed to mimic a living or deceased organism. The tissue may be healthy, diseased, and/or have genetic mutations. The term “natural tissue” or “biological tissue” and variations thereof as used herein refer to the biological tissue as it exists in its natural or in a state unmodified from when it was derived from an organism. A “micro-organ” refers to a segment of “bioengineered tissue” that mimics “natural tissue.”

The biological tissue may include any single tissue (e.g., a collection of cells that may be interconnected) or a group of tissues making up an organ or part or region of the body of an organism. The tissue may comprise a homogeneous cellular material or it may be a composite structure such as that found in regions of the body including the thorax which for instance can include lung tissue, skeletal tissue, and/or muscle tissue. Exemplary tissues include, but are not limited to those derived from liver, lung, thyroid, skin, pancreas, blood vessels, bladder, kidneys, brain, biliary tree, duodenum, abdominal aorta, iliac vein, heart and intestines, including any combination thereof.

The term “protein”, “peptide” and “polypeptide” are used interchangeably and in their broadest sense to refer to a compound of two or more subunit amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

The term “seeding” as used herein refers to a method of introducing cells onto something, for example, a biomatrix scaffold. Seeding may be carried out in a variety of containers, including but not limited to a plate and/or a bioreactor.

As used herein, the term “subject” is intended to mean any animal. In some embodiments, the subject may be a mammal; in further embodiments, the subject may be a human, mouse, or rat.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable.

Biomatrix Scaffolds and Extracellular Matrix

The term “extracellular matrix,” or “ECM,” as used herein, refers to the complex scaffold comprised of various biologically active molecules secreted by cells, adjacent to one or more cell surfaces, and involved in the structural and/or functional support of cells and tissues or organs comprised thereof. The chemical composition of the ECM is tissue-specific, though there are matrix components that are shared by many cell types. Specific matrix components and concentrations thereof may be associated with specific tissue types, histological structures, organs, and other super-cellular structures. Components of the extracellular matrix relevant to the instant disclosure include, but are not limited to collagens, collagen-associated matrix components (e.g. fibronectins, laminins, nidogen, elastins, proteoglycans, glycosaminoglycans), and signaling molecules (growth factors, cytokines).

In some embodiments, the primary considerations are the collagens and factors bound to them, since the strategy for isolating the biomatrix scaffolds is designed primarily to keep insoluble all collagen molecules of the tissue. In general, collagen molecules have an amino acid chemistry unique to each of the known collagen types. At present there are 29 types of collagens known. All known collagen molecules are comprised of 3 amino acid chains “woven” like a braid with a rod-shaped domain in the middle of the molecule and globular domains on either end (thus, causing the collagen molecules to have a “dumbbell”-like shape). The rod-shaped domains are dominated by repeats of a trio of amino acids, [glycine-proline-X], where X can be any amino acid. The globular domains are comprised of an amino acid sequence unique to each collagen type.

The collagens are secreted from cells and then one or both globular ends of the molecules are removed by specific peptidases followed by aggregation of multiple collagen molecules to form collagen fibrils. The exceptions are the “network collagens” (e.g. type IV or VI) that retain the globular domains and then aggregate end-on-end to form networks of collagen molecules with “chicken-wire”-like structures.

After aggregation into fibrils or into networks, the collagens are cross-linked through the effects of lysyl oxidase, an extracellular, copper-dependent enzyme that yields covalent bonding between collagen molecules (and also between elastin molecules) to produce cross-linked forms constituting very stable collagen molecular aggregates. The number of collagen molecules per fibril in the fibrillar collagens or the pattern of connections in the network collagens is dictated by the exact amino acid chemistry of the specific collagen type.

Extraction of a tissue to isolate its extracellular matrix is achievable with a strategy focused on isolation of a tissue's collagens in insoluble forms. The collagens are known to be the scaffolding to which non-collagenous matrix components attach, and signaling molecules bind to many of the matrix-bound components. The self-assembly of the complex of extracellular matrix components occurs with uncross-linked as well as cross-linked collagens. Thus, strategies to recover all of a tissue's collagens in an insoluble form is an ideal strategy for recovering most of the known components of the matrix.

The isolation of the matrix may be accomplished by utilizing buffers that are at neutral pH and with salt concentrations at or above 1 M. Whereas the cross-linked collagens can be isolated and preserved even with distilled water, the exact concentration of the salt required to preserve the uncross-linked collagens as insoluble depends on the collagen type. For example, Type I and III collagens, found in abundance in skin, require approximately 1 M salt to remain insoluble. By contrast the collagens in amniotic membranes with high levels of type V collagens require 3.5-4.5 M salt. The uncross-linked as well as cross-linked collagens in liver require approximately 3.4-3.5 M salt to remain insoluble.

Most methods of preparing extracts enriched in extracellular matrix make use of conditions that result in a loss of most, if not all, of the uncross-linked collagens and associated components. The most common strategies in matrix scaffold isolation use either a) enzymes that degrade matrix components and/or b) use of low salt or no salt buffers (e.g. distilled water) that result in dissolution of the uncross-linked collagens and any factors bound to them. Therefore, there are multiple forms of decellularized tissue extracts for matrix scaffolds that contain cross-linked collagens and any factors bound to those cross-linked collagens but are devoid of or have minimal amounts of the uncross-linked collagens and their associated components.

The term “biomatrix scaffold” (BMS) refers to an isolated extract of extracellular matrix produced by a strategy of keeping all of the tissue's collagens in an insoluble form. The BMS extracts are tissue-specific in their chemistry and in their effects. As described herein the BMS retains some, optionally most, of the collagens and/or collagen-bound factors found naturally in the biological tissue. In some embodiments the BMS comprises, consists of, or consists essentially of collagens, fibronectins, laminins, nidogen/entactins, elastins, integrins, proteoglycans, glycosaminoglycans (sulfated and non-sulfated—including hyaluronans) and any combination thereof, all being part of the biomatrix scaffold (e.g., encompassed in the term biomatrix scaffold).

In some embodiments, the BMS comprises a tissue's collagens that include (i) nascent (newly formed) collagens, (ii) aggregated but not cross-linked collagen molecules (collagen fibrils), (iii) cross-linked collagens, (iv) non-collagenous matrix components bound to collagens (e.g. laminins, fibronectins, nidogen/entactin, elastin, proteoglycans, glycosaminoglycans), (v), signaling molecules bound to these different forms of collagens and/or non-collagenous factors bound to collagens. In some embodiments, the vast majority of both cross-linked and uncross-linked native collagens found in the tissue along with non-collagenous matrix molecules and signaling molecules bound to these collagens. In some embodiments, the BMS comprises one or more collagen-associated matrix components such as laminins, nidogen, elastins, proteoglycans, hyaluronans, non-sulfated glycosaminoglycans, and sulfated glycosaminoglycans and growth factors and cytokines associated with the matrix components.

In some embodiments, the BMS comprises greater than 50% of matrix-bound signaling molecules found in vivo. In some embodiments, the matrix-bound signaling molecules may be epidermal growth factors (EGFs), fibroblast growth factors (FGFs), hepatocyte growth factors (HGFs), insulin-like growth factors (IGFs), bone morphogenetic factors (BMFs), transforming growth factors (TGFs), interleukins (IL), nerve growth factors (NGFs), neurotrophic factors, leukemia inhibitory factors (LIFs), vascular endothelial cell growth factors (VEGFs), platelet-derived growth factors (PDGFs), stem cell factor (SCFs), colony stimulating factors (CSFs), GM-CSFs, erythropoietin, thrombopoietin, heparin binding growth factors, IGF binding proteins, placental growth factors, and Wnt signals.

In some embodiments, the BMS disclosed herein is prepared avoiding low ionic strength buffers to preserve both the cross-linked and the non-cross-linked collagens. In some embodiments, the BMS may lack a detectable amount of specific collagens, fibronectins, laminins, nidogen/entactins, elastins, proteogylcans, glycosaminoglycans and/or any combination thereof. In some embodiments essentially all of the collagens and collagen-bound factors are retained. In other embodiments the BMS comprises all of the collagens known to be in the tissue.

The BMS may comprise at least about 50%, 60%, 70%, 75%, 80%, 85%, 90%, 95%, 97%, 98%, 99%, 99.5% or 100% of the collagens, collagen-associated matrix components, and/or matrix bound signaling molecules (e.g. growth factors, hormones and/or cytokines), in any combination, found in the natural biological tissue. In some embodiments the BMS comprises at least 95% of the collagens and most of the collagen-associated matrix components and matrix-bound signaling molecules of the biological tissue. The collagens described herein may be nascent (newly formed), collagens that are aggregated to form fibrils but still not cross-linked, and some may be cross-linked forms of these. Exemplary collagens and methods of extraction thereof are described in brief herein below.

In some embodiments, the biomatrix scaffolds disclosed herein contain essentially all of the collagens comprising the nascent (newly formed) collagens, the aggregated collagen molecules that self-assemble to form fibrils prior to cross-linking, plus the cross-linked collagens. In addition, the biomatrix scaffold may optionally comprise other matrix components plus signaling molecules that are bound to these collagens or to bound matrix components. In some embodiments, the ratio of collagens in the biomatrix scaffold is similar or identical to the ratio in the tissue from which the biomatrix scaffold is derived. Non-limiting examples of a suitable percentage of nascent collagens and aggregated uncross-linked collagens to mimic the original tissue include, but are not limited to, at least about or about 0.05%, 0.1%, 0.5%, 1%, 5%, 10%, 15%, 20%, 25%, 30%, 35%, 40%, 45%, or 50%.

As described herein, most of the collagen-associated matrix components and matrix bound growth factors, hormones and/or cytokines of the biological tissue” refers to the biomatrix scaffold retaining about 50%, 60%, 70%, 75%, 80%, 85%, 90%, 95%, 97%, 98%, 99%, 99.5% or 100% of the collagen-associated matrix components and matrix bound growth factors, hormones and/or cytokines found in the natural (e.g., unprocessed) biological tissue. The terms “powdered” or “pulverized” are used interchangeably herein to describe a biomatrix scaffold that has been ground into a powder. The term “three-dimensional biomatrix scaffold” refers to a decellularized scaffold that retains its native three dimensional structure. Such three-dimensional scaffolds may be either a whole scaffold or frozen sections thereof. For some purposes, scaffolds can be pulverized at liquid nitrogen temperatures, a process called cryopulverization.

Exemplary collagens include any and all types of collagen, such as those currently identified as type I through type XXIX collagens, but not limited to these, thus allowing for future recognition of yet other types of collagens. The biomatrix scaffold may comprise at least about 50%, 60%, 70%, 75%, 80%, 85%, 90%, 95%, 97%, 98%, 99%, 99.5% or more of one or more of the collagens found in the native biological tissue. In some embodiments the collagens are cross-linked and/or uncross-linked. The amount of collagen in the biomatrix scaffold can be determined by various methods known in the art and as described herein, such as but not limited to determining the hydroxyproline content.

Exemplary methods of determining whether the cross-linked or uncross-linked character of a collagen also exist, such as those that rely on observing its dissolution properties. See e.g. D. R. Eyre,* M. Weis, and J. Wu. Advances in collagen cross-link analysis Methods, 2009; 45 (1): 65-74 (describing analysis of cross-linking by standard methods in the field of collagen chemistry). For example, a collagen may be determined to be cross-linked based on whether it dissolves in buffers at or below 1 M salt concentration.

Exemplary collagen-associated matrix components include, but are not limited to, adhesion molecules (the families of fibronectins and laminins); L- and P-selectin; heparin-binding growth-associated molecule (HB-GAM); thrombospondin type I repeat (TSR); amyloid P (AP); nidogens/entactins; elastins; vimentins; proteoglycans (PGs); chondroitin sulfate PGs (CS-PGs); dermatan sulfate-PGs (DS-PGs); members of the small leucine-rich proteoglycans (SLRP) family such as biglycan and decorins; heparin-PGs (HP-PGs); heparan sulfate-PGs (HS-PGs) such as glypicans, syndecans, and perlecans; and glycosaminoglycans (GAGs) such as hyaluronans, heparan sulfates, chondroitin sulfates, keratan sulfates, and heparins.

In some embodiments the biomatrix scaffold comprises, consists of, or consists essentially of collagens, fibronectins, laminins, nidogens/entactins, elastins, proteoglycans, glycosaminoglycans (GAGs), growth factors, hormones, and cytokines (in any combination) bound to various matrix components. The biomatrix scaffold may comprise at least about 50%, 70%, 75%, 80%, 85%, 90%, 95%, 97%, 98%, 99%, 99.5% or more of one or more of the collagen-associated matrix components, hormones and/or cytokines found in the natural biological tissue and/or may have one or more of these components present at a concentration that is at least about 50%, 70%, 75%, 80%, 85%, 90%, 95%, 97%, 98%, 99%, 99.5% or more of that found in the natural biological tissue.

In some embodiments the biomatrix scaffold comprises all or most of the collagen-associated matrix components, hormones and/or cytokines known to be in the tissue. In other embodiments the biomatrix scaffold comprises, consists essentially of or consists of one or more of the collagen-associated matrix components, hormones and/or cytokines at concentrations that are close to those found in the natural biological tissue (e.g., about 50%, 60%, 70%, 75%, 80%, 85%, 90%, 95%, 98% or 100% of the concentration found in the natural tissue).

Exemplary matrix-bound signaling molecules include, but are not limited to, bone morphogenetic proteins (BMPs), epidermal growth factors (EGFs), fibroblast growth factors (FGFs), hepatocyte growth factors (HGFs), insulin-like growth factors (IGFs), transforming growth factors (TGFs), nerve growth factors (NGFs), neurotrophic factors, leukemia inhibitory factors (LIFs), vascular endothelial cell growth factors (VEGFs), platelet-derived growth factors (PDGFs), stem cell factor (SCFs), colony stimulating factors (CSFs), GM-CSFs, erythropoietin, thrombopoietin, heparin binding growth factors, IGF binding proteins, placental growth factors, Wnt signals.

Exemplary cytokines include, but are not limited to interleukins, lymphokines, monokines, colony stimulating factors, chemokines, interferons and tumor necrosis factor (TNF). The biomatrix scaffold may comprise at least about 50%, 60%, 70%, 75%, 80%, 85%, 90%, 95%, 97%, 98%, 99%, 99.5%, 100% or more (in any combination) of one or more of the matrix bound growth factors and/or cytokines found in the natural biological tissue and/or may have one or more of these growth factors and/or cytokines (in any combination) present at a concentration that is at least about 50%, 60%, 70%, 75%, 80%, 85%, 90%, 95%, 97%, 98%, 99%, 99.5%, 100% or more of that found in the natural biological tissue.

In some embodiments the biomatrix scaffold comprises physiological levels or near-physiological levels of many or most of the matrix bound growth factors, hormones and/or cytokines known to be in the natural tissue and/or detected in the tissue and in other embodiments the biomatrix scaffold comprises one or more of the matrix bound growth factors, hormones and/or cytokines at concentrations that are close to those physiological concentrations found in the natural biological tissue (e.g., differing by no more than about 30%, 25%, 20%, 25%, 20%, 15%, 10%, 5%, 4%, 3%, 2%, 1%, 0.5% in comparison). The amount or concentration of growth factors or cytokines present in the biomatrix scaffold can be determined by various methods known in the art and as described herein, such as but not limited to various antibody assays and growth factor assays.

Methods and compositions relating to biomatrix scaffolds and isolation thereof are discussed in, for example, U.S. Pat. Nos. 8,802,081, 9,102,913, 7,456,017, and U.S. Provisional Application No. 62/335,013.

Biomatrix scaffolds, as disclosed herein may be used in various forms including, but not limited to, a frozen section of biomatrix scaffold, a cryopulverized biomatrix scaffold, or an intact biomatrix scaffold.

Cancers and Tumors

As used herein, the term “cancer” refers to cancer originating from any part of the body or any cell type. This includes, but is not limited to, carcinoma, sarcoma, hemangioma, lymphoma, leukemia, germ cell tumors, and blastoma. In some embodiments, the cancer is associated with a specific location in the body or a specific disease. As used herein, the term “tumor” refers to any type of tumor tissue, benign or malignant. It is well understood that cancer therapy and/or chemotherapeutic agents may be used to treat certain tumors.

In general, cancers are diseases of tissues and involving genetic and/or epigenetic aberrations affecting cell-cell relationships within the tissue/organ. Among the most common are aberrations of the epithelial-mesenchymal cell relationship constituting a fundamental cell-cell relationship within metazoans, organisms comprised of tissues, organized communities of cells. All normal tissues are comprised of maturational lineages of epithelial cells partnered with maturational lineages of mesenchymal cells and with the lineages being coordinate with each other in maturational processes. The epithelial-mesenchymal relationship is mediated by paracrine signaling consisting of dynamic and synergistic interactions of extracellular matrix complexes and signaling molecules. One or more mutations of cells, caused by an inherited genetic defect or by radiation or by an environmental toxin (chemicals) can result in qualitative or quantitative independence of the epithelial cells from the mesenchymal cells. Carcinomas are malignancies of epithelia; sarcomas are malignancies of the mesenchymal cells; hemangiomas are ones of endothelia; leukemias and lymphomas are representative of malignancies of blood cells; etc. Malignant cells can occur in any tissue in a donor of any age and can cause disruption at the primary site, the site at which the malignancy first occurs. The malignancy can spread, i.e. metastasize to other sites within the body and cause disruption in the distant sites. In some embodiments, the cancer is associated with a specific location in the body or a specific disease.

Malignant transformation of cells involves one or more genetic and/or epigenetic changes in cells and that are specific to particular tumor types. A discussion of known genetic and epigenetic changes in malignancy and especially in metastatic potential is given in numerous recent reviews. See, e.g., Turajlic S, Swanton C. Metastasis as an evolutionary process. Science. 2016 Apr. 8; 352(6282):169-75. Review; Suvà M L, Riggi N, Bernstein B E. Epigenetic reprogramming in cancer. Science. 2013 Mar. 29; 339(6127):1567-70. Review; Vanharanta S, Massagué J. Origins of metastatic traits. Cancer Cell. 2013 Oct. 14; 24(4):410-21. Review; Vogelstein B1, Papadopoulos N, Velculescu V E, Zhou S, Diaz L A Jr, Kinzler K W. Cancer genome landscapes. Science. 2013 Mar. 29; 339(6127):1546-58. Review; Marquardt J U, Factor V M, Thorgeirsson S S. Epigenetic regulation of cancer stem cells in liver cancer: current concepts and clinical implications. Journal of Hepatology. 2010 September; 53(3):568-77. Review.

The presence of one or more genetic or epigenetic changes giving rise to malignant transformation can result in alterations in the interactions of the malignant cells with neighboring cells. The malignant cells can become less dependent on paracrine signals that normally coordinate the activities of the cellular community of the tissue. In recent years it has been found that some of the changes involve “exosomes”, plasma membrane-encapsulated circles that are blebbed from the malignant cells. The exosomes can be released into the interstitial fluid, into blood, and/or into the microenvironment of neighboring cells. The exosomes contain portions of the malignant cells' cytoplasmic components, microRNAs, and enzymatic activities, can fuse with the plasma membranes of neighboring cells, and so deliver the contents of the exosome into the neighboring cells; this process can alter the biological activity of the neighboring cells. The exosomes can also be distributed via the lymphatics or blood stream to sites distant from that of the primary tumor, fuse with cells in those sites, and alter the biological activity of cells in those distant sites. Indeed, this modification of neighboring cells or cells at a distant site can be a prequel to invasion or to metastasis.

The lethal aspect of most malignancies is their ability to metastasize. The ability of tumors to spread to distant sites has long been known to demonstrate patterns in where the cells go. Breast and prostate cancers metastasize to bone, liver, lung and brain; colon cancers metastasize to liver, lung and peritoneum; thyroid cancers spread to liver, lungs and bone; etc. Although early stages of metastasis involve a restricted set of tissues with metastatic lesions, the late stages of cancers involves spread throughout the body.

Variables influential to the process of metastasis include the production of enzymes by the tumor cells and that can dissolve components of the extracellular matrix enabling invasion and dispersing of the tumor cells to distant sites. The repertoire of enzymes is distinct in different categories of tumors. For example, sarcomas produce enzymes that allow tumor cells to spread quickly into the blood vessels and so facilitate spread of the tumors to other sites by hematogenous (vascular) routes. By contrast, carcinomas typically produce enzymes that enable tumor cells to spread into lymphatic channels and only at late stages into vascular channels.

The route of spread or metastasis (e.g. hematogenous versus lymphatic routes) leads secondarily to the seeding of tumor cells into diverse tissues. Even when tumor cells have spread to and are found attached into a tissue, they do not necessarily grow and colonize that tissue. At early and intermediate stages in cancers, the tumors preferentially will grow or colonize only certain distant sites. By contrast, at late stages of cancer, the tumor cells usually can be found in most tissues, having overwhelmed whatever “barriers” there are to growth of the tumor cells within those tissues.

This organ-site specificity of metastasis was described in 1889 by Stephen Paget, who referred to the phenomena as the “seed and soil” hypothesis. By this, Dr. Paget meant that there are variables in the “seed” (the tumor cells) and in the “soil” (the microenvironment of a tissue). It has been the subject of investigations for more than a hundred years as summarized in a recent review by Dr. Isaiah Fidler (The pathogenesis of cancer metastasis: the ‘seed and soil’ hypothesis revisited. Nature Reviews Cancer 2003; 3, 453-458)

Although the repertoire of enzymes produced by a given tumor is known to be a variable in the route of spread and to some extent also in the ability to colonize a given tissue, that repertoire of enzymes is not sufficient to explain the phenomena of organ-site specificity of metastasis. Applicants show in the experiments below that another set of the variables is in the chemistry of the tissue-specific forms of extracellular matrix. The chemistry of the matrix is unique to each tissue and dictates the ability of the tumor cells to survive and grow in the tissue's microenvironment. Not to be bound by theory, Applicants hypothesize that some of the variables that Paget ascribed to the “soil” are those attributable to the extracellular matrix. At early to intermediate stages of the disease, the matrix chemistry's control can dominate and dictate whether or not the tumor cells survive and grow; at late stages, the tumor cells produce so much (and so many) enzymes that the variables within the matrix and controlling tumor cell growth are weakened or destroyed resulting in the ability of the tumor cells to grow in most tissues.

Cancer Therapies and Chemotherapeutic Agents

As used herein, the term “cancer therapy” intends any known treatment regimen used for cancer, including but not limited to cryotherapy, hyperthermia, photodynamic therapy, laser therapy, radiation therapy, cancer-specific antibody therapy, chemotherapy, adoptive cell transfer, cytokine therapy, immunotherapies, vaccination, Bacillus Calmette-Guérin (BCG), CAR cell therapy, endocrine therapy (also known as hormone therapy), stem cell therapy (autologous, allogenic, or syngenic), and other types of targeted or untargeted therapy. See National Cancer Institute website at www.cancer.gov, last visited May 6, 2016.

As used herein, the term “chemotherapeutic agent” refers to a moiety useful to treat cancer, such as a small molecule chemical compound used to treat cancer, and encompasses all dosage forms, formulations, and regiments of known agents useful to treat cancer. Non-limiting examples of a chemotherapeutic agent include an anthracycline, such as doxorubicin, daunorubicin, epirubicin, idarubicin, valrubicin, or a derivative thereof; an antibiotic, such as actinomycin-D, bleomycin, mitomycin-C, or a derivative thereof; an alkylating agent, such as cyclophosphamide, mecholrethamine, uramustine, melphalan, chlorambucil, ifosfamide, bendamustine, carmustine, lomustine, streptozocin, busulfan, dacarbazine, temozolomide, thiotepa, altretamine, or a derivative thereof; a platinum-based agent, such as cisplatin, carboplatin, nedaplatin, oxaliplatin, satraplatin, triplatin tetranitrate, or a derivative thereof; an anti-metabolite, such as 5-fluorouracil, 6-mercaptopurine, capecitabine, cladribine, clofarabine, cystarbine, floxuridine, fludarabine, gemcitabine, hydroxyurea, methotrexate, pemetrexed, pentostatin, thoguanin, or a derivative thereof; a topoisomerase inhibitor, such as camptothecin, topotecan, irinotecan, etoposide, teniposide, mitoxantrone, or a derivative thereof; a mitotic inhibitor, such as paclitaxel, docetaxel, izabepilone, vinblastine, vincristine, vindesine, vinorelbine, estramustine, or a derivative thereof.

Cytoreductive agents, such as agents that act to reduce cellular proliferation, are known in the art and widely used. Those agents that kill cancer cells only when they are dividing are termed “cell-cycle specific” and include agents that act in S-phase (e.g. topoisomerase inhibitors and anti-metabolites).

Toposiomerase inhibitors are drugs that interfere with the action of topoisomerase enzymes (topoisomerase I and II). During the process of chemo treatments, topoisomerase enzymes control the manipulation of the structure of DNA necessary for replication, and are thus cell cycle specific. Examples of topoisomerase I inhibitors include the camptothecan analogs listed above, irinotecan and topotecan. Examples of topoisomerase II inhibitors include amsacrine, etoposide, etoposide phosphate, and teniposide.

Anti-metabolites are usually analogs of normal metabolic substrates, often interfering with processes involved in chromosomal replication. They attack cells at very specific phases in the cycle. Anti-metabolites include folic acid antagonists, e.g., methotrexate; pyrimidine antagonist, e.g., 5-fluorouracil, foxuridine, cytarabine, capecitabine, and gemcitabine; purine antagonist, e.g., 6-mercaptopurine and 6-thioguanine; adenosine deaminase inhibitor, e.g., cladribine, fludarabine, nelarabine and pentostatin; and the like.

Plant alkaloids are derived from certain types of plants. The vinca alkaloids are made from the periwinkle plant (Catharanthus rosea). The taxanes are made from the bark of the Pacific Yew tree (taxus). The vinca alkaloids and taxanes are also known as antimicrotubule agents. The podophyllotoxins are derived from the May apple plant. Camptothecan analogs are derived from the Asian “Happy Tree” (Camptotheca acuminata). Podophyllotoxins and camptothecan analogs are also classified as topoisomerase inhibitors. The plant alkaloids are generally cell-cycle specific.

Examples of these agents include vinca alkaloids, e.g., vincristine, vinblastine and vinorelbine; taxanes, e.g., paclitaxel and docetaxel; podophyllotoxins, e.g., etoposide and tenisopide; and camptothecan analogs, e.g., irinotecan and topotecan.

As used herein, the term “endocrine therapy” refers to all methods of endocrine therapy, i.e. treatment that adds, blocks, or removes hormones. For certain conditions (such as diabetes or menopause), hormones are given to adjust low hormone levels. For certain conditions, to slow or stop the growth of certain cancers (such as prostate and breast cancer), synthetic hormones or other drugs may be given to block the body's natural hormones. See NCI Dictionary of Cancer Terms.

As used herein, the term “immunotherapy” refers to a type of biological therapy that uses substances to stimulate or suppress the immune system to help the body fight cancer, infection, and other diseases. Some types of immunotherapy only target certain cells of the immune system. Others affect the immune system in a general way. Non-limiting examples of immunotherapy include cytokines, vaccines, bacillus Calmette-Guerin (BCG), and some monoclonal antibodies. See NCI Dictionary of Cancer Terms. In some embodiments, the immunotherapy may be dendritic cell therapy, antibody-dependent therapy, T-cell dependent therapy, or NK-cell dependent therapy

As used herein, the term “molecular therapy” refers to a personalized therapy designed to treat cancer by interrupting unique molecular abnormalities that drive cancer growth. Drugs and/or molecular agents—such as, but not limited to, inhibitory or antisense oligonucleotides (e.g. any type of interfering RNA, locked nucleic acids (LNA), etc.)—can be used in targeted therapy designed to interfere with a specific biochemical pathway central to the development, growth, and spread of the particular cancer.

As used herein, the term “radiation therapy” refers to all methods of radiation therapy, including external beam radiation therapy, sealed source ration therapy, and systemic radioisotope therapy. In some embodiments, the radiation is focused locally to the target site, such as to a tumor site. In some embodiments, radiation therapy is effected prior to administration of the prodrug conjugate. In any embodiments using radiation therapy, the radiation therapy may include gamma-knife radiation, cyber-knife radiation, and/or high intensity focused ultrasound radiation.

The phrase “first line” or “second line” or “third line” refers to the order of treatment received by a patient. First line therapy regimens are treatments given first, whereas second or third line therapy are given after the first line therapy or after the second line therapy, respectively. The National Cancer Institute defines first line therapy as “the first treatment for a disease or condition. In patients with cancer, primary treatment can be surgery, chemotherapy, radiation therapy, or a combination of these therapies. First line therapy is also referred to those skilled in the art as “primary therapy and primary treatment.” See National Cancer Institute website at www.cancer.gov, last visited May 6, 2016. Typically, a patient is given a subsequent chemotherapy regimen because the patient did not show a positive clinical or sub-clinical response to the first line therapy or the first line therapy has stopped.

### Abbreviations

The following abbreviations appear throughout the present disclosure.

The following is a non-limiting list of abbreviations used herein and independent of those used for the growth factors and cytokines: If an acronym indicates a factor and is given in Italics, it refers to a gene; if in regular font, it indicates a protein encoded by the gene. If an acronym is for a cell population, the species from which the cell derived is indicated by a small letter in front of the acronym. For example, m=murine (mouse); r=rat; h=human.

BMS, biomatrix scaffold, a tissue-specific extract enriched in extracellular matrix; Caco-2, epithelial colorectal-adenocarcinoma cell line established by Dr. Jorgen Fogh (Sloan-Kettering Cancer Institute, NYC, N.Y.)—it has intestinal stem cell properties that enable it under distinct conditions to lineage restrict either into small intestine-like cells versus into large intestine (colon) cells depending on the culture conditions; CD, common determinant; CD34, hemopoietic stem/progenitor cell antigen; CD45, common leucocyte antigen found on most hemopoietic cell subpopulations; CRC, colorectal cancer; CYP, cytochrome P450 mono-oxygenases that catalyze many reactions associated with drug metabolism and/or synthesis of cholesterol, steroids and lipids; CK, cytokeratin; CK7, cytokeratin associated with biliary cells; CK8 and CK18, cytokeratins associated with all epithelia; EpCAM, epithelial cell adhesion molecule; FBS, fetal bovine serum; GAGs, glycosaminoglycans, carbohydrate chains that are polymers of a dimer (uronic acid and an aminosugar), most of them have specific sulfation patterns that play diverse roles cooperatively with proteins in signal transduction processes; HDL, high-density lipoprotein; HDM, a serum-free, hormonally defined medium used for maintenance or differentiation of specific cell types; H&E, hematoxylin and eosin; HT-29, a colorectal adenocarcinoma cell line behaving as undifferentiated cells under some conditions whereas under differentiation conditions yields cells with a polarized morphology, characterized by the redistribution of membrane antigens and development of an apical brush-border membrane; KM, Kubota's Medium, a serum-free medium designed for endodermal stem cells and with some modifications can also be used for maturational descendants of the stem cells such as hepatoblasts or committed progenitors; LGR5, Leucine-rich repeat-containing G-protein coupled receptor 5, an important stem cell marker in intestine, liver and pancreas; MMP, matrix metalloproteinase (or peptidase); MMP2, matrix metalloproteinase-2, the 72 kDa type IV collagenase or gelatinase A (GELA); MMP9, matrix metallopeptidase 9, also known as 92 kDa type IV collagenase or gelatinase B (GELB), is a matrixin, a class of enzymes of the zinc-metalloproteinases family involved in degradation of the extracellular matrix; PAS, Periodic acid—Schiff; SDC, sodium deoxycholate; SEM, scanning electron microscopy; SW480, a human colorectal adenocarcinoma cell line derived from a metastatic lesion (disease: Duke's Type B); TEM, transmission electron microscopy.

### Acronyms for Signals (Growth Factors, Hormones, Cytokines)

BMPs, bone morphogenetic proteins are multi-functional growth factors that belong to the transforming growth factor beta (TGF-beta) superfamily; bFGF, basic fibroblast growth factor; EGF, epidermal growth factor; EGFR, epidermal growth factor receptor; FGFs, fibroblast growth factors (e.g. FGF-4, FGF-6, FGF-7); FGF-4, fibroblast growth factor-4; FGF-6, fibroblast growth factor-6; FGF-7, fibroblast growth factor-7; GCSF, granulocyte-colony stimulating factor; GDNF, glial-derived neurotrophic factor; GM-CSF, granulocyte macrophage-colony stimulating factor; hGH, human growth hormone; HGF, hepatocyte growth factor; HB-EGF, heparin-binding epidermal growth factor; IGFBP-1, insulin-like growth factor binding protein 1; IGFBP-3, insulin-like growth factor binding protein 3; IGFBP-4, insulin-like growth factor binding protein 4; IGFBP-6, insulin-like growth factor binding protein 6; IGF-I, insulin-like growth factor-I; IGF-1 SR, insulin-like growth factor-I receptor; IGF-II, insulin-like growth factor-II; IL, Interleukin (e.g. IL-6, IL-11); M-CSF, macrophage-colony stimulating factor; M-CSF R, macrophage-colony stimulating factor receptor; NT-3, neurotrophin-3; NT-4, neurotrophin-4; PDGF R a, platelet-derived growth factor receptor alpha; PDGF R b, platelet-derived growth factor receptor beta; PDGF-AA, platelet-derived growth factor AA; PDGF-AB, platelet-derived growth factor AB; PDGF-BB, platelet-derived growth factor BB; PIGF, phosphatidylinositol glycan anchor biosynthesis class F; SCF, stromal cell-derived factor-1; SCF R, stromal cell-derived factor receptor; TGF-α, transforming growth factor alpha; TGF-β, transforming growth factor beta; TGF-β2, transforming growth factor beta 2; TGF-β3, transforming growth factor beta 3; VEGF, vascular endothelial growth factor; VEGF R2, vascular endothelial growth factor receptor 2; VEGF R3, vascular endothelial growth factor receptor 3; VEGF-D, vascular endothelial growth factor receptor D.

### Modes of Carrying Out the Disclosure

Aspects of this invention relate to methods, kits, and compositions related to diagnosing and/or characterizing a cancer or tumor. In any of the embodiments described relating to the cancer or tumor herein, the cancer or tumor may be malignant. In certain embodiments, the cancer or tumor may be characterized by its potential to metastasize to specific tissues and/or the responsiveness of one or more of such metastases to respond to one or more cancer therapies and/or chemotherapeutic agents described herein below.

Method embodiments disclosed herein comprise seeding one or more biomatrix scaffolds with cells from a tumor or cancer.

In some of these method embodiments, the cells may be from (1) cells of a tumor biopsy or sample taken from the patient diagnosed with a cancer or tumor or (2) cells from cell line of the same cancer or tumor type as that of the patient. In some of these method embodiments, the cells are analyzed for their ability to form a colony of growing cells. In some embodiments, the colony is on the substratum of a biomatrix scaffold. In some of these method embodiments, the cells form a colony. In some of these method embodiments, the cells are analyzed for their ability to become 3-dimensional colony of growing cells. In some of these method embodiments, the cells form a 3-dimensional colony of growing cells.

In some of these method embodiments, the cells are analyzed for their ability to express genes or to secrete proteins or other factors. Non-limiting examples of these genes include: pluripotency genes (e.g. OCT4, SOX2, KLF4, KLF5, SALL4, NANOG, BMi-1), stem cell genes (e.g. EpCAM, LGR5/LGR6, CXCR4, one or more of the many variants of the CD44 family of genes encoding hyaluronan receptors, multidrug resistance genes (e.g. mdr gene family, p-glycoproteins), genes encoding enzymes that dissolve extracellular matrix components (e.g. hyaluronidase, collagenases, elastase, matrix degrading metalloproteinases); non-limiting examples of these proteins include: the proteins encoded by the genes noted above (e.g. CD44, p-glycoproteins, the matrix degrading enzymes); non-limiting examples of other factors relevant to this analysis include exosome production, microRNAs, qualitative or quantitative independence of paracrine signaling from mesenchymal feeders. In some embodiments of these method embodiments, this analysis may occur once the cells have been seeded and attach onto one or more biomatrix scaffolds. In some embodiments of these method embodiments, the cells may attach to the substratum of tissue-specific forms of the one or more biomatrix scaffolds.

In some of these method embodiments, the one or more biomatrix scaffolds originating from one or more predetermined organs, respectively, of a human body. In further embodiments, the predetermined organs are selected based on the cancer or tumor type of the patient—e.g. to include organs known to be associated with metastases of the cancer or tumor type or to include the organ in which the cancer or tumor was found. In all method embodiments, each biomatrix scaffold recapitulates the biological facets of the tissue from which it originated.

The seeding method may optionally be used to determine a potential of a tumor to metastasize within a patient diagnosed with a cancer or tumor. For example, metastasis into an organ may be predicted if the tumor or cancer cell forms a colony of growing cells on a biomatrix scaffold. The predicted metastases may be further determined to be localized to organ from which said scaffold was derived. Thus, in some embodiments, metastasis into one or more predetermined organs is predicted in vivo if the cells form a colony of growing cells on the one or more biomatrix scaffolds, respectively, originating therefrom.

The seeding method may also be used to determine the appropriate treatment for a tumor in a patient diagnosed with a cancer or tumor. This may be done in addition to determining a potential of a tumor to metastasize or independently therefrom.

Any of the above disclosed method embodiments may further comprise characterizing the colony based on its histology, exosome production, microRNA production, interactions with mesenchymal cells, and/or gene expression profile.

Any of the above disclosed method embodiments may further comprise screening the colony for responsiveness to one or more cancer therapies and/or chemotherapeutic agents or treating the colony with one or more cancer therapies and/or chemotherapeutic agents. Such cancer therapies and/or chemotherapeutic agents include, but are not limited to, one or more doses of radiation therapy, immunotherapy, endocrine therapy, molecular therapy, and/or one or more of an anthracycline, an alkylating agent, a platinum-based agent, an anti-metabolite, a topoisomerase inhibitor, or a mitotic inhibitor. Such cancer therapies and/or chemotherapeutic agents may optionally be selected based on the cancer or tumor type of the patient. In certain embodiments, the responsiveness of the cancer or tumor to the one or more therapies and/or agents in vivo in an organ may be correlated with the responsiveness of said the cancer or tumor cells to the one or more therapies or agents when seeded on a biomatrix scaffold of the organ.

Aspects of the disclosure also relate to kits comprising one or more biomatrix scaffolds originating from one or more predetermined organs, respectively, of a human body and instructions to carry out one or more of the methods disclosed herein above. Further embodiments contemplate kits further comprising one or more cancer therapies and/or chemotherapeutic agents; tools or instructions for obtaining cells of a tumor biopsy or sample taken from the patient diagnosed with a cancer or tumor; cells from cell line from a particular cancer or tumor type; and/or any reagents, media, or other components required to carry out the above disclosed methods.

Further aspects of the disclosure contemplate an artificial tumor or metastases model specific to one or more predetermined organs of a human body generated by the seeding method disclosed herein above.

### EXAMPLES

The following examples are non-limiting and illustrative of procedures which can be used in various instances in carrying the disclosure into effect. Additionally, all references disclosed herein below are incorporated by reference in their entirety.

### Example 1—Generation of Biomatrix Scaffolds and Characterizations

All animal experiments were in accordance with guidelines provided by University of North Carolina Institutional Animal Care and Use Committee.

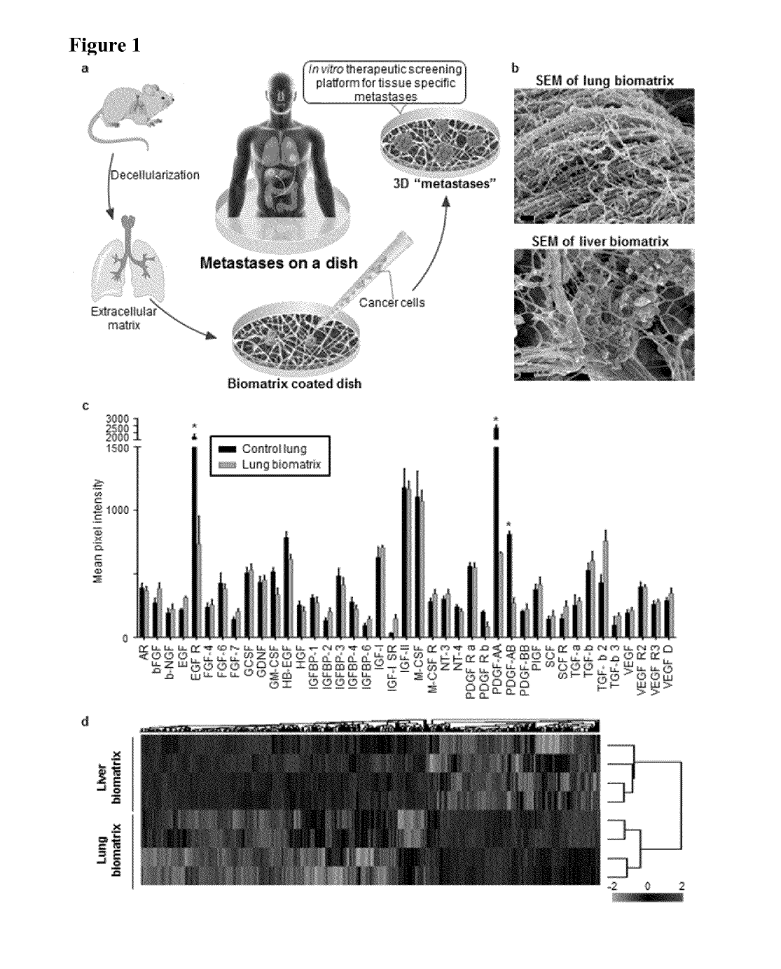

A breakthrough in the field of tissue engineering has been the use of extracellular matrix extracts prepared by tissue decellularization methods, especially those done by perfusion protocols. Decellularization protocols are ones in which an organ or tissue is chemically stripped of its cells, leaving behind an extracellular matrix extract with a chemical composition similar to that in vivo. Importantly, decellularization preserves the complex composition of extracellular matrices and even their anatomical features found in normal organs, which would be nearly impossible to recreate using synthetic techniques. Applicants hypothesized that matrix extracts from decellularized tissues could be used to create a tissue-specific in vitro culture platform to engineer cancer “metastases” (FIG. 1a). While previous studies have successfully used a variety of methods to decellularize tissues and engineer complex organs, including liver and lung, the degree to which cell signaling molecules are preserved using these methods remains largely unknown⁷. There is one exception, the protocol for preparation of “biomatrix scaffolds” was shown to retain physiological levels of all known signaling molecules found in liver tissue¹². However, it was not known if the set of signaling molecules preserved in such biomatrix scaffolds is tissue-specific. Applicants utilized the protocol for preparation of biomatrix scaffolds (BMSs) that retains >98% of the tissue's matrix components and preserves physiological levels of matrix-bound growth factors and cytokines. As proof of concept, Applicants used this culture platform to study metastatic CRC cells. Given that liver and lung are the most common sites of metastasis in CRC patients, Applicants aimed to engineer in vitro liver and lung metastases.

### Biomatrix Scaffolds Prepared by Perfusion-Based Decellularization of Liver and Lung

Sprague-Dawley rats (male, 250-300 g) were used to produce liver and lung biomatrix scaffolds using a protocol established previously. Biomatrix scaffolds (BMSs) were prepared by cannulating the portal vein (liver BMS) or inferior vena cava (lung BMS) for perfusion with decellularization reagents. The vasculature was perfused with basal medium (e.g. serum-free DMEM/F12) until blood was eliminated and then with 250 mLs of 1% sodium deoxycholate (SDC) containing 36 units/L phospholipase. Next, organs were rinsed with serum-free basal medium and then perfused with 3.5 M NaCl (prepared basal medium) until the perfusate was negative for proteins as assessed by optical density (OD 280.) Finally, the BMS samples were rinsed with basal medium and snap frozen. Frozen BMS samples were pulverized into a fine powder using a freezer mill (Spex SamplePrep 6770, Metuchen, N.J.). Processed BMS powder was stored at −80° C.

Preparation of Tissue Culture Plates Coated with Biomatrix Scaffold Powder

The BMS samples were dissolved in a solution composed of 4 M guanidine HCl, 50 mM sodium acetate (pH 5.8), and 25 mM EDTA containing proteinase and phosphatase inhibitor cocktails. BCA assays were then performed to determine total protein concentrations. Medium (DMEM/F12) containing BMS samples was added to tissue culture plates or onto Nunc Thermanox coverslips (Thermofisher Scientific, Waltham, Mass.) and allowed to dry overnight. Plates were sterilized using 100 Gy of external beam irradiation (Precision X-Ray, Inc, North Branford, Conn.).

### Growth Factors Antibody Array

Tissues (lungs, livers) and BMS samples from those tissues were sent to RayBiotech (Norcross, Ga.) where they were processed and submitted for growth factor array analysis. Specifically, the relative levels of growth factors and cytokines were quantified by the RayBio Human Growth Factor Antibody Array G-Series 1 (Cat #AAH-GF-G1-8). Data were expressed in normalized signal intensities.

### Mass Spectrometry Analysis

Lung BMSs (n=4) and liver BMSs (n=4) were pulverized, and protein was extracted and purified as previously described22. Each sample (50 μg) was reduced with 5 mM DTT, alkylated with 15 mM iodoacetamide, and digested with trypsin (Promega, Madison, Wis.) overnight at 37° C. The peptide samples were desalted using C18 spin columns (Pierce). The peptide samples (1 μg) were analyzed by LC/MS/MS using an Easy nLC 1000 coupled to a QExactive HF mass spectrometer (Thermo Scientific, Waltham, Mass.). and separated over a 2 hr method. The gradient for separation consisted of 5-32% mobile phase B at a 250 nl/min flow rate, where mobile phase A was 0.1% formic acid in water and mobile phase B consisted of 0.1% formic acid in ACN. The QExactive HF was operated in data-dependent mode where the 15 most intense precursors were selected for subsequent fragmentation. Resolution for the precursor scan (m/z 400-1600) was set to 120,000 with a target value of 3×10⁶ ions. MS/MS scans resolution was set to 15,000 with a target value of 5×10⁴ ions. The normalized collision energy was set to 27% for HCD. Peptide match was set to preferred, and precursors with unknown charge or a charge state of 1 and ≥7 were excluded.

Raw data files were processed using MaxQuant version 1.5.3.17 and searched against a Uniprot rat database (downloaded December 2016, containing 29,795 entries), using Andromeda within MaxQuant. Enzyme specificity was set to trypsin, up to two missed cleavage sites were allowed, carbamidomethylation of Cys was set as a fixed modification and oxidation of Met was set as a variable modification. A 1% false discovery rate (FDR) was used to filter all data. Label-free quantification using razor+unique peptides and match between runs (1 min time window) were enabled. A minimum of 3 unique peptides per protein was required for quantification. Proteins with >60% missing values were removed. Statistical analysis was performed in Perseus version 1.5.6.0 using ANOVA with p<0.05 considered significant. Hierarchical clustering using the z-score normalized LFQ intensities of the significant proteins was performed.

### Cell Culture

Human colorectal cancer cell lines (HT-29, Caco2 and SW480) were acquired from the Tissue Culture Facility at UNC. The luciferase-expressing cell line, HT-29-luc2, was purchased from Caliper Life Sciences (Hopkinton, Mass.). Cell lines were authenticated using short tandem repeats and were tested for mycoplasma contamination. HT-29, HT-29-luc2, and SW480 cells were cultured in DMEM/F12 (Gibco, Invitrogen, Carlsbad, Calif.) supplemented with 10% fetal bovine serum (Gibco) and penicillin/streptomycin (Mediatech, Manassas, Va.). Caco2 cells were cultured in DMEM/F12 (Gibco) supplemented with 20% fetal bovine serum (Gibco) and penicillin/streptomycin (Mediatech). Cells were passaged on normal tissue culture plates or tissue culture plates coated with collagen, Matrigel, liver BMSs, and lung BMSs (100 μg/cm²).

### Cell Seeding Efficacy

CRC cells were seeded at plates coated with collagen, Matrigel, liver BMSs, and lung BMSs (100 ug/cm²). After 24 h, cultures were washed with PBS and lysed in 500 μL of DNA lysis solution. DNA concentrations were assessed using a Qubit dsDNA BR Assay Kit.

### Cellular Growth Rates

CRC cells were grown on plastic, collagen, Matrigel, liver BMSs, or lung BMSs (100 ug/cm²) in 6-well plates. Cells were collected at various time points post seeding and placed in DNA lysis solution. DNA concentrations were assessed using a Qubit dsDNA BR Assay Kit. Growth rate over time was standardized based on seeding efficiencies.

### Assessment of Proliferation and Apoptosis In Vitro

For proliferation assays, CRC cells grown on plastic, collagen, Matrigel, liver BMSs, or lung BMSs (100 μg/cm²) were incubated with 10 μM 5-ethynyl-2′-deoxyuridine (EdU) for 4 hours. Cells were then washed with PBS, processed into single cells using TrypLE, and stained for EdU using a Click-iT Plus EdU Assay for Flow Cytometry kit (Cat. no. C10646) according to the manufacturer's instructions. Cells were then washed in PBS containing 10% FBS three times and submitted for flow cytometric analysis.

For apoptosis assays, CRC cells grown on plastic, collagen, Matrigel, liver BMSs, or lung BMSs (100 μg/cm²) were collected, processed into single cells, and fixed in 4% paraformaldehyde for 10 minutes at room temperature. Cell suspensions were blocked over night in Dako block (cat). Cells were then stained with primary conjugated Cleaved Caspase 3 (1:100) for 2 hours at room temperature. washed with PBS containing 10% FBS three times, and submitted for flow cytometric analysis. All flow cytometric analysis was done using a Beckman Coulter CyAn ADP and analyzed using software Summit 5.2.

### Anoikis Assays

CRC cells grown on plastic, collagen, Matrigel, liver BMSs or lung BMSs were seeded at 1×10⁴/well to the Anchorage Resistance Plate (Cell Biolabs) or a control 96-well cell culture plate. Cells were allowed to culture for 48 h. Live cells were detected with Calcein AM and the fluorescence was read using a microplate reader.

### Invasion Assay

CRC cells grown on plastic, collagen, Matrigel, liver BMSs, or lung BMSs in serum free media were placed into the upper chamber of an insert coated with Matrigel (Corning). The lower chamber contained DMEM/F12 with 10% fetal bovine serum. After 16 h culture, non-invading cells were removed by a cotton swab and the cells on the lower surface of the membrane were fixed with 100% methanol and stained with 1% Toluidine Blue. Cells were counted in five fields using an inverted microscope.

### Scanning Electron Microscopy (SEM)

Cover slips were coated with collagen, Matrigel, liver BMS or lung BMS. Cultures grown on these substrata were fixed in a 0.15 M sodium phosphate buffer (pH 7.4) solution containing 3% glutaraldehyde overnight at 4° C. Samples were rinsed with PBS three times and then dehydrated using serial incubations in increasingly concentrated ethanol solutions (30%, 50%, 75% to 100%) for 10 min each. Cover slips were transferred to a Samdri-795 critical point dryer and dried using CO2 as the transitional solvent (Tousimis Research Corporation, Rockville, Md.). The coverslips were then mounted on 13 mm aluminum planchets with double sided carbon adhesive tabs and sputter-coated with 10 nm of gold palladium alloy (60Au:40Pd, Hummer X Sputter Coater, Anatech USA, Union City, Calif.). Images were acquired using a Zeiss Supra 25 FESEM operating at 5 kV, with working distance of 5 mm, and 10 um aperture (Carl Zeiss Microscopy, Pleasanton, Calif.).

### Transmission Electron Microscopy (TEM)

Cell colonies were fixed in 0.15M sodium phosphate buffer (pH 7.4) solution containing 3% glutaraldehyde for 1 h at room temperature. Samples were then rinsed with PBS and post-fixed with 1% osmium tetroxide/1.25% potassium ferrocyanide/0.15M PBS for 1 h. Following post-fix, samples were rinsed with deionized water and dehydrated using serial incubations in increasingly concentrated ethanol solutions (30%, 50%, 75% to 100%) for 10 min each (30%, 50%, 75%, to 100%). Samples were then incubated in a 1:1 mixture of propylene oxide/Polybed 812 epoxy resin (Polysciences, Inc., Warrington, Pa.) overnight followed by 100% resin for 24 hours. Samples were then placed in fresh Polybed 812 epoxy resin and sectioned transversely at 70 nm using a diamond knife and a Leica Ultracut UCT microtome (Leica Microsystems). Sections were mounted on mesh copper grids and stained with 4% aqueous uranyl acetate and Reynold's lead citrate. The grids were observed at 80 kV using a LEO EM910 transmission electron microscope (Carl Zeiss SMT, LLC). Images were acquired using a Gatan Onus SC 1000 CCD Camera with DigitalMicrograph 3.11.0 software (Pleasanton, Calif.).

### Histology

Cell colonies were fixed in 4% paraformaldehyde for 1 h at room temperature, paraffin embedded, and cut into 4 μm sections. Sections were stained with hematoxylin and eosin (H&E).

### Intrasplenic Injection

Athymic Nu/Nu mice (male, 8-10 weeks old) were obtained from the animal colony at UNC. Mice were anesthetized with an intraperitoneal injection of ketamine (100 mg/kg) and dexdomitor (1 mg/kg). An incision was made on the left side of the abdomen to expose the spleen. Then 5×106 HT-29 or HT-29-luc2 cells in 50 ul PBS suspension per mouse were injected intrasplenically. All animal experiments were approved by the UNC Institutional Animal Care and Use Committee.

### Gene Expression Microarray

Total RNA was extracted using RNeasy Mini Kit (Qiagen) according to manufacturer's instructions from HT-29 cells grown on plastic, Matrigel, and liver BMSs. RNA was also isolated from liver metastases generated following intrasplenic injection of HT-29 cells. Four biological replicates of cells grown under each condition were used. RNA quality was evaluated by Agilent Bioanalyzer 2100. RNA samples were sent to Lineberger Comprehensive Cancer Center Genomics Core at University of North Carolina at Chapel Hill. Samples were analyzed using Agilent SurePrint G3 Unrestricted Gene Expression 8x60K Microarray (human G4858A). The cDNA was labeled with Cy3-CTP prior to hybridization.

Microarray data analyses were performed using GeneSpring 12.6 GX software. Raw signal values were quantile normalized, and probe sets were filtered based on flag values. Normalized intensity values were performed hierarchical clustering analysis using Euclidean distance to sort both entities and samples. Differently expressed genes were selected based on statistical (one way ANOVA p<0.05) and fold change (>=4) thresholds. Genes that were upregulated in both engineered liver metastasis and in vivo metastasis were analyzed using gene ontology. Data have been submitted to the Gene Expression Omnibus database (accession number GSE76180).

Real-time PCR

Total RNA was isolated from CRC cells grown on plastic, collagen, Matrigel, and liver BMSs using the RNeasy mini kit (QIAGEN, Valencia, Calif.). Total RNA was reverse transcribed into cDNA using the Quantitek cDNA synthesis kit (QIAGEN). Quantification of Timp1 transcript was conducted by the 2^{-ΔΔCt} method using β-Actin as the normalizer. The sequence of primers used for Timp1 gene expression was as follows: forward primer 5′-AGACCTACACTGTTGGCTGTGAG-3′; reverse primer 5′-GACTGGAAGCCCTTTTCAGAG-3′.

### Drug Response Assays

CRC cells were seeded at 2×104 cells/well in 96-well plates coated with collagen, Matrigel, liver BMSs and lung BMSs (100 μg/cm2). One day post seeding, cells were treated with chemotherapeutics for 24 hours. Chemotherapeutics were then removed, and cells remained in culture for another 24 hours in standard culture media. Cell viability was determined by MTS [(3-(4,5-dimethylthiazol-2-yl)-5-(3-carboxymethoxyphenyl)-2-(4-sulfophenyl)-2H-tetrazolium)] cell proliferation assays using the CellTiter 96® Aqueous One Solution Cell Proliferation assay kit (Promega, Madison, Wis.). Treatment responses for each culture condition were standardized to untreated cultures.

### Clonogenic Assays

Plating efficiency (PE) of each cell line was determined. Cells grown on plastic, collagen, Matrigel, liver BMSs, and lung BMS were irradiated with 0, 2, 4, 6, and 8 Gy. Following irradiation, cells were plated into 25 mL flasks at densities ranging from 100 to 250,000. Cells were incubated for 14 days, fixed, and then stained a solution composed of 4% formaldehyde, 80% methanol, and 0.25% crystal violet. Only colonies containing 30 or more cells were counted. The surviving fraction (SF) was calculated using the formula: (# of colonies formed)/(# of plated cells) (Plating Efficiency). The SF was plotted against the radiation dose on a log scale. Linear-quadratic formula SF=e\hat{ }(−αD−βD2) was used to generate survival curves using the R package “CFAssay”.

### In Vivo Assessment of Metastatic Potential

HT-29-luc2 cells grown on plastic, collagen, Matrigel, liver BMSs, or lung BMSs were trypsinized, harvested and processed into single cell suspensions. For hypoxic cell culture, HT-29-luc2 cells were cultured in a hypoxia incubator chamber with 1% 02 and 5% CO2 with 37° C. for 24 hours. Cells were suspended in PBS and administered into athymic Nu/Nu mice (male, 8-10 weeks old) via tail vein injection (1×10⁶/mouse) or via direct hepatic injection (2×10³/mouse). Bioluminescence was measured every week using an IVIS imaging system (Caliper). Mice were randomly assigned to groups. Cell injection and bioluminescence imaging were not blinded to investigators. No animals were excluded from statistical analysis. Luciferase intensity for each time point was normalized to the respective intensity value at day 0. Lung and liver from the sacrificed mice were removed and examined by ex vivo bioluminescence.

### Statistical Analysis

Data analysis was performed using either GraphPad Prism 6 (GraphPad, La Jolla, Calif.) or R statistical software. Results from cell growth rate, seeding efficiency, MTS, flow cytometry, anoikis assay, invasion assay and real-time PCR, were analyzed by one-way ANOVA followed by Tukey's Honestly Significant Difference post-hoc test. For the clonogenic assay, linear-quadratic cell survival curves were analyzed using R package “CFAssay”. No statistical methods were used to predetermine sample size. F-test was used to test the equality of two variances. P-values less than 0.05 were considered significant.

### Data Availability

Microarray data are available in Gene Expression Omnibus under the accession number for the study: ncbi.nlm.nih.gov/geo/query/acc.cgi?token=sdmluyaqplgvtuz&acc=GSE76180

### Discussion

Metastasis is the main cause of morbidity and mortality in cancer patients and understanding the biology of metastasis can lead to significant improvements in cancer treatment. One of the hallmarks of cancer metastasis is organ-specificity. Each type of cancer has a unique pattern of metastatic spread that cannot be explained entirely by organ proximity or sites dictated by vascular flow. While it is generally accepted that normal tissue microenvironments play an important role in modulating metastatic growth and development, the biology underlying this interaction is poorly understood. This is due largely to the lack of experimental models that are easy to use and that fully recapitulate the biology of organ-specificity in cancer metastasis. Existing in vitro/ex vivo model systems do not possess organ-specificity due to an absence of components present in the organ-microenvironment. Although collagen and Matrigel can be used to provide a partially three-dimensional (3D) culture substratum, the composition of these substrata are highly dissimilar from the tissue-specific microenvironments in vivo and encountered by metastases. Although genetically engineered animal models that develop metastases in vivo can be used to study metastatic cancer in a tissue-specific manner, they are costly and difficult to use. Understanding the biology underlying the interaction between metastases and the tissue microenvironment they inhabit may lead to more effective cancer treatments for metastatic cancer patients, as prior studies have determined that the treatment response of metastases can differ between metastatic sites.

Therefore, there is a strong need for in vitro models that can recapitulate in vivo biology of cancer metastasis. Such models can facilitate the development of effective therapies for cancer metastases. This is especially important given that treatment responses of metastases are known to differ between metastatic sites and can differ from that of the primary tumor²⁻⁵. Hence, we aimed to develop a novel in vitro cancer metastases model that possesses organ-specificity.

The organ microenvironment is hypothesized to be a critical component to incorporate into the development of organ-specific metastases models. Given that matrix extracts prepared by decellularization processes have been utilized to bioengineer complex organs, it was theorized that these matrix extracts produced by one or another of the existing protocols might prove to yield excellent substrata for engineering cancer metastases⁶⁻⁸ (FIG. 1a). However, the only method for tissue decellularization that retains tissue specificity in terms of chemical composition as well as functionally is that described by Rojkind and Reid, matrix extracts referred to as “bomatrices.”

### Organ Specific Biomatrix Scaffolds Recapitulate the In Vivo Biochemical Environment

A highly improved version of the Rojkind-Reid protocol was developed by Wang et al. and shown to yield a matrix extract, referred to as “biomatrix scaffolds”, that retain >98% of the tissue's collagens and collagen-associated matrix components (e.g. fibronectins, laminins, elastin, proteoglycans, etc.). In addition, the biomatrix scaffolds have been shown to contain all known signaling molecules (growth factors, cytokines) bound to any of the tissue's matrix components and at levels similar to those found in vivo. The matrix components and the bound signals were shown to be retained in histologically accurate locations. Biomatrix scaffolds were prepared from rat livers using the Wang et al protocol. Employing a similar protocol, lung biomatrix scaffolds were prepared.

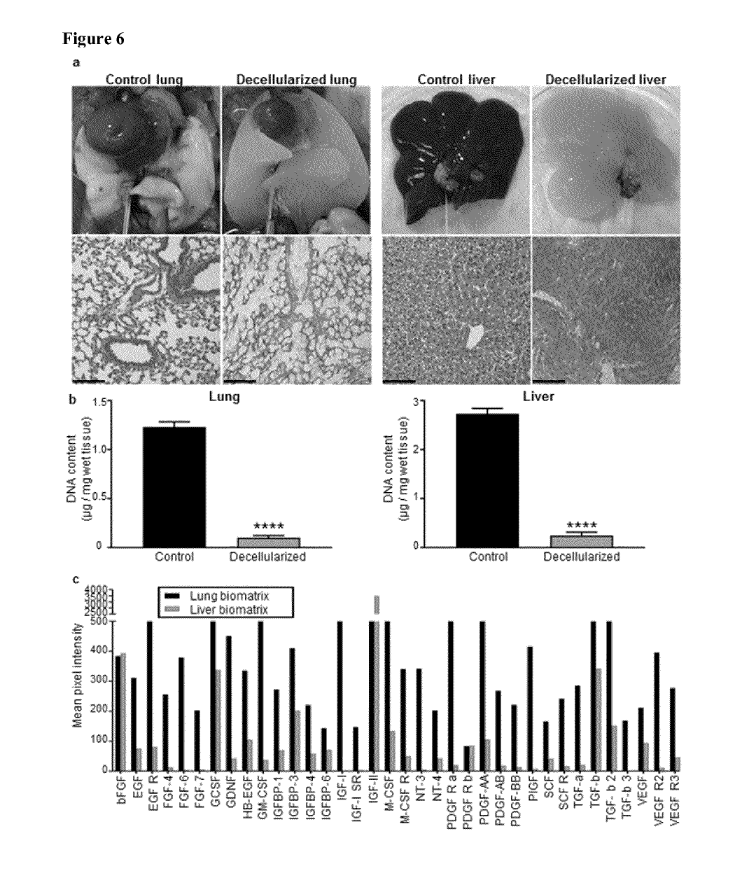

The rat's inferior vena cava (IVC) was cannulated for the infusion of decellularization reagents and the superior vena cava (SVC) was clamped using a vessel clip. An opening was made in the rat's carotid artery for outflow. The color change of the rat lung (from white to nearly transparent) provided a preliminary indication of successful decellularization FIG. 6a). Decellularized liver BMSs were prepared by cannulating the hepatic portal vein for the infusion of decellularization reagents (FIG. 6b). Complete decellularization was confirmed histologically and by assessing nucleic acid content of the BMS material (Supplemental FIG. 1a,b). Notably, these BMSs naturally formed a meshwork of fibrous proteins and carbohdyrates that completely coated tissue culture plates (FIG. 1b).

To assess whether lung BMSs contained signaling molecules present within the in vivo lung microenvironment, we evaluated the relative abundance of growth factors and cytokines retained by our liver BMSs following decellularization using semi-quantitative ELISA. In agreement with previous data demonstrating that extracellular matrix bound signaling molecules are retained following liver decellularization¹², lung biomatrix scaffolds retained almost all (93%) of the analyzed growth factors and cytokines at near physiologic levels (FIG. 1c). Note that the relative abundance of these signaling molecules varies between liver and lung BMSs consistent with their tissue specific nature (FIG. 6c).

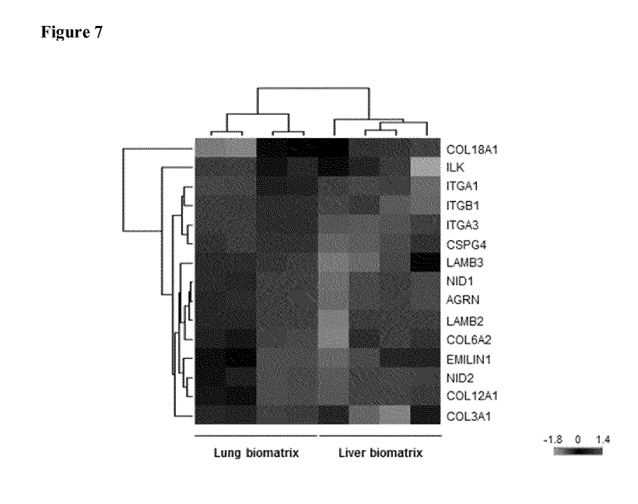

To further evaluate molecular differences present between liver and lung BMSs, we performed a mass spectrometric analysis. As with extracellular matrix-bound growth factors and cytokines, we found that the relative composition of the extracellular matrix itself also differed between liver and lung BMSs (FIG. 1d; FIG. 7).

### CRC Cell Lines Form Liver and Lung “Metastases” In Vitro

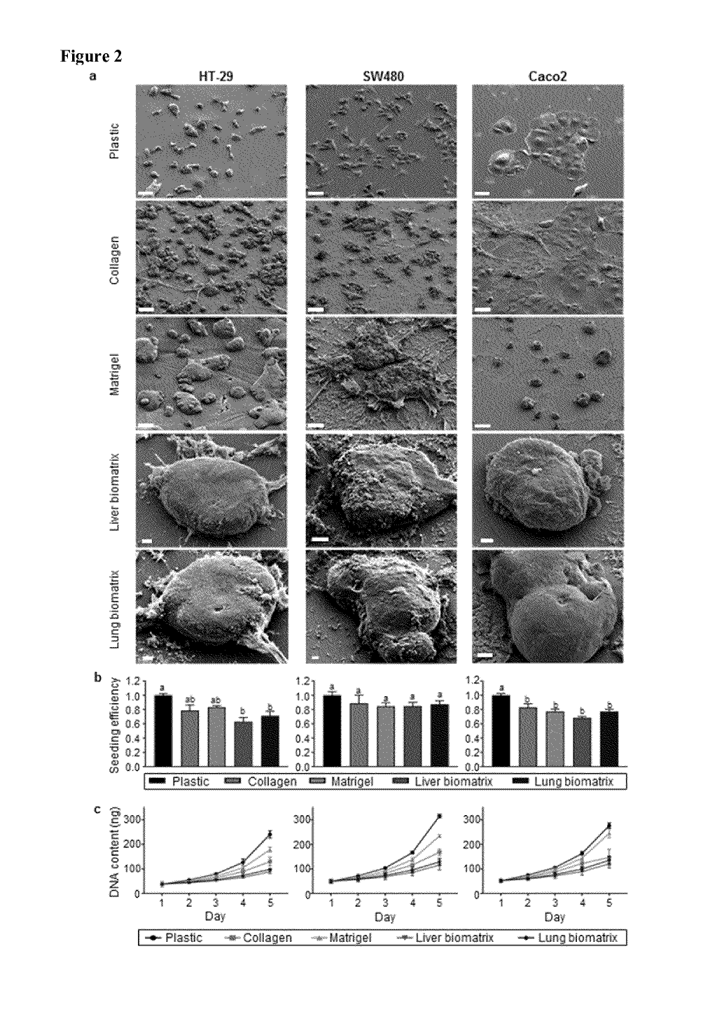

To engineer tissue-specific CRC cancer metastases, we cultured CRC cell lines (HT-29, SW480, and Caco2) on tissue culture dishes coated with liver and lung BMSs. Excitingly, all three CRC cell lines spontaneously formed three-dimensional (3D) spheroid colonies comprised of tumor cells bound together via tight junctions (FIG. 2a; FIG. 7a,b). These “metastases” are relatively large in scale, attaining diameters of up to a millimeter. Tumor spheroids that attain a diameter of greater than 500 micrometers contain necrotic cores due to a general lack of oxygen and nutrient availability as well as the internal accumulation of cytotoxic metabolites. Consistent with this observation, metastases engineered on BMSs also contain necrotic regions similar to the hypoxic and necrotic regions found in in vivo metastases (FIG. 7c).

To characterize the behavior of our engineered metastases, we compared the seeding efficiencies and growth rates of CRC cells grown on liver and lung BMSs to that of cells grown on plastic, collagen, and Matrigel. Applicants found that CRC cells demonstrated reduced seeding efficiencies on collagen, Matrigel, liver BMS, and lung BMS when compared to cells grown on plastic (FIG. 2b). Furthermore, Applicants found that cells grown on collagen, liver BMS, and lung BMS grow more slowly than cells grown on plastic and Matrigel (FIG. 2c). The slower growth rate observed in the engineered metastases is consistent with the behavior of metastatic cancer cells in vivo.

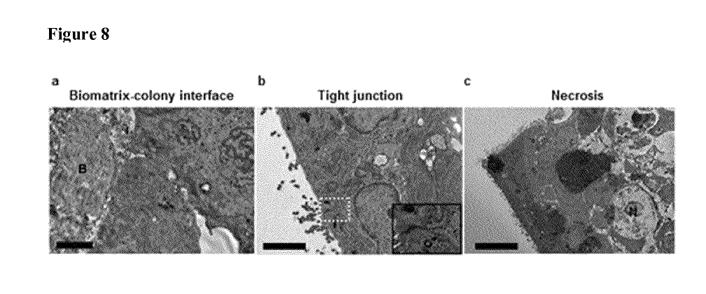

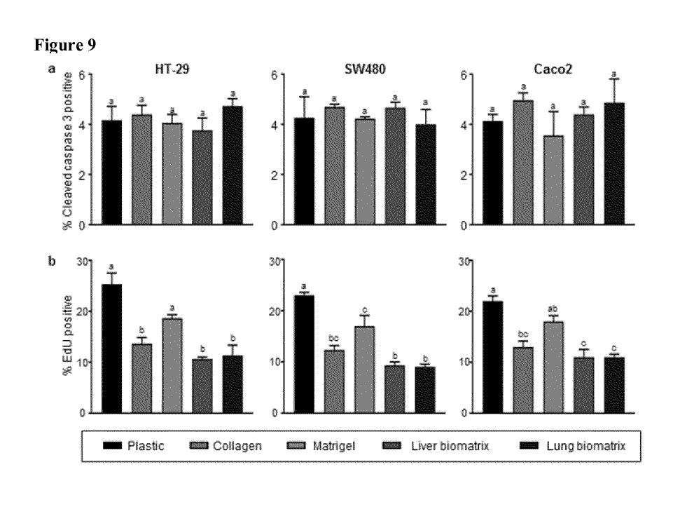

Applicants next sought to determine whether the relatively slow growth rate displayed by liver and lung engineered metastases is due to increased rates of apoptosis or reduced rates of proliferation. To characterize apoptosis in cancer cells grown on different substrata, Applicants assessed the expression of Cleaved Caspase 3, a marker of apoptosis, using flow cytometry. Applicants found that CRC cells in all culture conditions exhibited comparable rates of apoptosis (FIG. 9a). To assess the relative proliferation rates of cancer cells grown on different substrata, we performed an EdU cell proliferation assay. Applicants incubated cultures with EdU, a thymidine analog that is incorporated into DNA as cells enter S-phase, for four hours. Subsequently, Applicants quantified the number of cells that underwent S-phase (EdU positive) using flow cytometry. Applicants found that tumor cells grown on liver and lung biomatrix scaffolds demonstrated the lowest proliferation rates of all culture conditions tested across all three cell lines (FIG. 8b). Cumulatively, these data demonstrate that CRC cells seeded on BMSs generate relatively large slowly growing cultures that are more comparable to in vivo metastases than cultures produced by other culture platforms.

### Engineered Liver Metastases Closely Resemble Metastases Found In Vivo

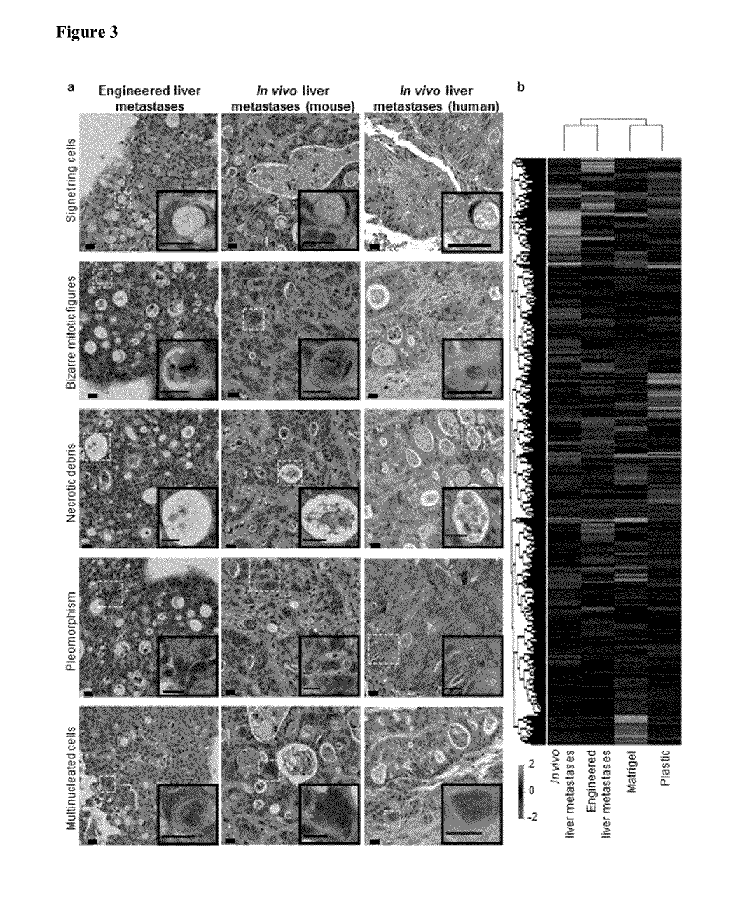

To further assess the degree of similarity that cancer cells grown on BMSs and conventional substrata share with in vivo metastases, Applicants conducted a histopathological analysis. The histology of cells grown on plastic, collagen, Matrigel, and liver biomatrices was compared with in vivo liver metastases present in nude mice and liver metastases from patients with metastatic CRC cancer. Classic histological features of liver metastases of gastrointestinal origin found in vivo include: (1) signet ring cells, (2) bizarre mitotic figures, (3) necrotic debris (extracellular accumulations of eosinophilic and nuclear debris), (4) pleomorphic cell size and shape, and (5) multinucleated cells. Applicants were able to identify all of these features in the engineered liver metastases generated from HT-29, SW480, and Caco2 CRC cells (FIG. 3a; FIG. 8). In contrast, CRC cells grown on collagen and Matrigel only demonstrated bizarre mitotic figures and multinucleated cells and CRC cells grown on standard plastic culture dishes contained none of these histological features (FIG. 3a; FIG. 8). Signet ring cells are an in vivo pathologic finding that has not been reported in ex vivo model systems. These data demonstrate that engineer metastases contain the same pathologic features found in in vivo liver metastases.

| | | Signet ring | Bizzare mitotic | Necrotic | | Multinucleated |
|---|---|---|---|---|---|---|
| | | cells | figures | debris | Pleomorphism | cells |
| HT-29 | Plastic | | | | | |
| | Collagen | | X | | | X |
| | Matrigel | | X | | | X |
| | Liver biomatrix | X | X | X | X | X |
| | Lung biomatrix | X | | X | X | X |
| SW480 | Plastic | | | | | X |
| | Collagen | | | | | X |
| | Matrigel | | | | | X |
| | Liver biomatrix | X | X | X | X | X |
| | Lung biomatrix | X | | X | X | X |
| Caco2 | Plastic | | X | | X | |
| | Collagen | | X | | X | X |
| | Matrigel | | X | | X | |
| | Liver biomatrix | X | X | X | | X |
| | Lung biomatrix | X | X | X | | X |

In addition to exploring the histologic characteristics shared by cancer cells grown on different substrata and in vivo metastases, Applicants also sought to investigate the degree of similarity between their respective transcriptomes. Specifically, Applicants compared the global gene expression profiles of HT-29 cells cultured on plastic, Matrigel, and liver BMSs to in vivo liver metastases formed via splenic injection of HT-29 cells (FIG. 9a,b). Hierarchical clustering analysis revealed that the gene expression signature of our engineered liver metastases is more comparable to in vivo liver metastases than to HT-29 cells grown on plastic or Matrigel (FIG. 3b). The relatively high degree of similarity shared by engineered metastases and in vivo metastases was further assessed by comparing gene expression profiles of engineered metastases and CRC cells grown on Matrigel to cells grown on plastic.

A total of 791 genes were observed to be up-regulated in both bioengineered metastases and in vivo liver metastases when compared to cells grown on plastic. Many of these commonly up-regulated genes were found involved in biological responses to oxidative stress and hypoxia.

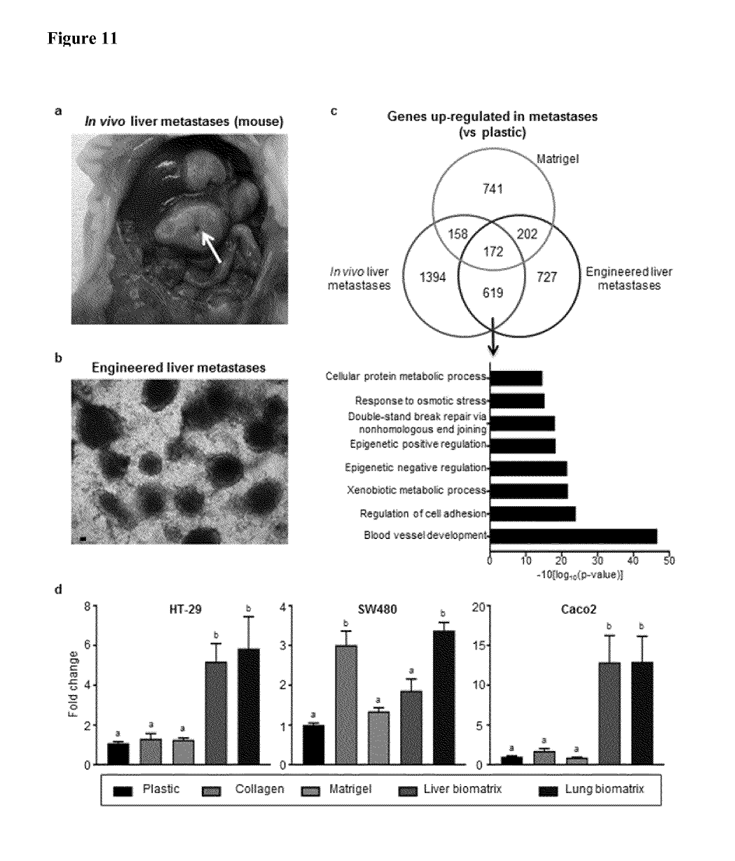

A total of 619 genes were observed to be discretely up-regulated in engineered metastases and in vivo liver metastases when compared to cells grown on plastic and Matrigel (FIG. 11c). The commonly up-regulated genes were found to be associated with the functions angiogenesis, cellular adhesion, and drug metabolism (FIG. 11c).

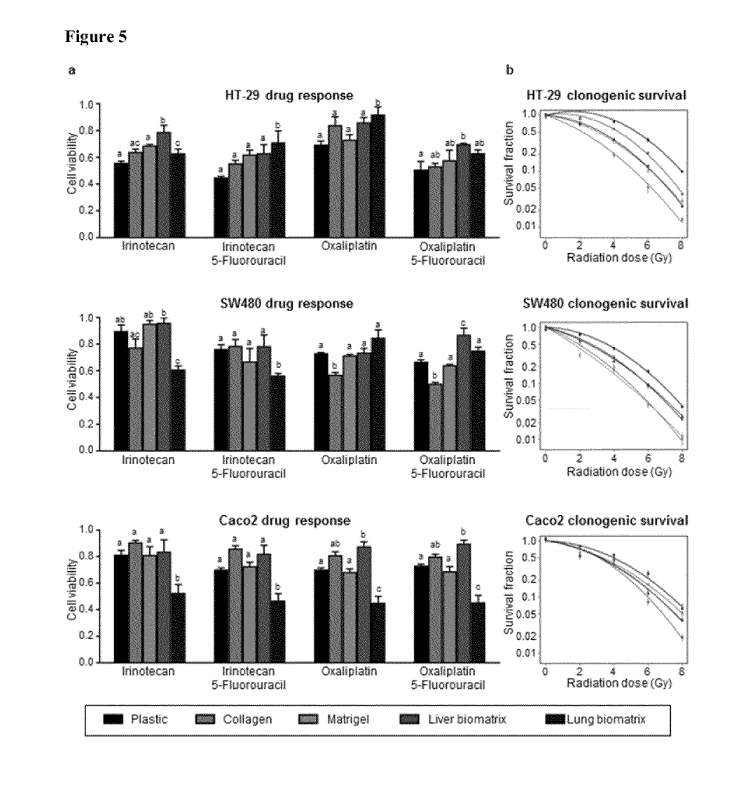

Applicants also observed that in vivo and engineered liver CRC metastases express higher levels of Timp1 than CRC cells cultured Matrigel and plastic (Supplemental FIG. 5d). This finding is consistent with previous studies demonstrating higher expression of TIMP1 in liver metastases than in the primary tumors of CRC patients. Taken together, these data demonstrate that the engineered metastases closely mimic in vivo metastases phenotypically and biologically.

### Engineered Metastases Demonstrate Increased Metastatic Potential In Vivo

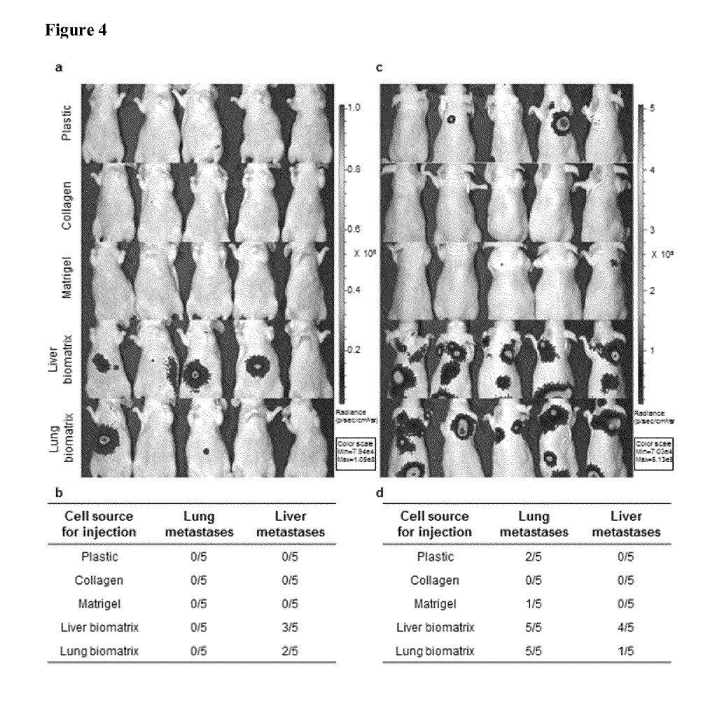

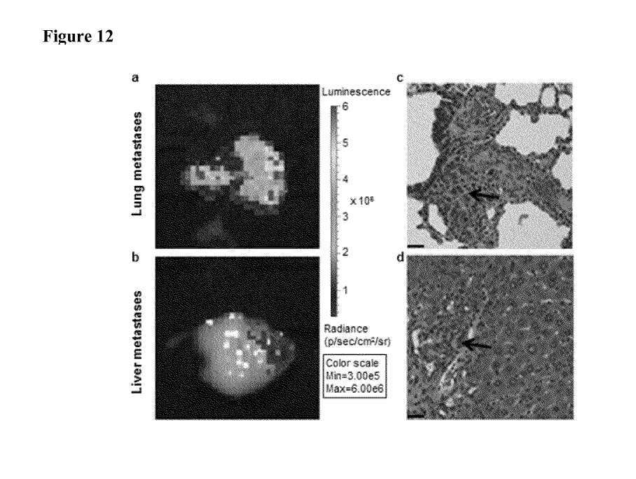

Applicants sought to determine whether the histological and molecular similarities that engineered metastases share with in vivo metastases would functionally translate to an increased ability to grow within respective in vivo tissues. Namely, Applicants hypothesized that if BMSs recapitulate tissue specific microenvironments, then cells grown on BMSs should be able survive and grow in their corresponding in vivo microenvironments. For example, cells grown on liver BMSs should be more adept to grow in liver tissue. To test this hypothesis, Applicants delivered HT-29-luc2 cells grown on plastic, collagen, Matrigel, liver BMSs, and lung BMSs to the livers and lungs of hosts and subsequently assessed their ability to form metastases in vivo using bioluminescent imaging (FIG. 4a,c; FIG. 12). To determine the relative ability of engineered liver metastases to grow in liver tissue, Applicants used direct hepatic injections to deliver CRC cells grown in different culture conditions to the liver and found that cells isolated from engineered liver metastases were more capable of forming liver metastases in vivo than cells grown on plastic, collagen, Matrigel, or lung BMSs (FIG. 4a,b, FIG. 11).

To assess the relative ability of engineered lung metastases to grow in lung tissue, we delivered CRC cells grown on different substrata to the lung using tail vein injection. Applicants found that cells grown on lung BMSs demonstrated a higher capacity to form lung metastases than cells grown on all conventional culture substrata (FIG. 4c,d; FIG. 12). Unexpectedly, Applicants found that cells grown on liver BMSs can also form lung metastases. Moreover, Applicants found that cells from liver BMSs and to a lesser extent lung BMSs formed liver metastases following tail vein injection (FIG. 4a,b; FIG. 12).

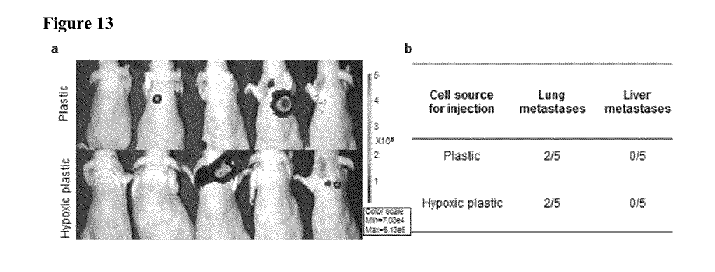

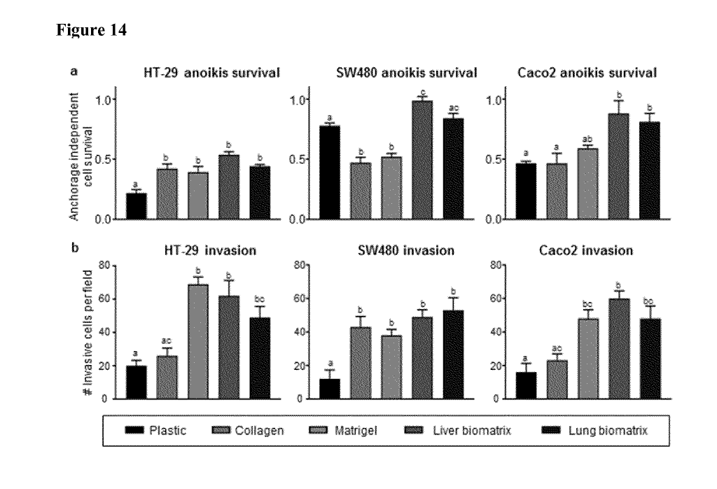

Cancer cells exposed to hypoxic conditions can demonstrate enhanced survival and resistance to undergoing apoptosis. To determine if the increased lung metastatic potential displayed by engineered metastases is attributable to the presence of hypoxic cells, Applicants delivered cells grown on plastic in hypoxic conditions to the lung using tail vein injection. Applicants found that hypoxic preconditioning of CRC cells grown on plastic did not enhance their capacity to form lung metastases (FIG. 13). Additionally, cells delivered using tail vein injection must be able to avoid anoikis and colonize lung tissue prior to developing lung metastases. Importantly, Applicants found that the increased lung metastatic potential displayed by HT-29 cells grown on liver and lung BMSs was not attributable to either increased resistance to anoikis or enhanced invasion ability (FIG. 14a,b). Taken together, these data demonstrate that engineered metastases are more capable of growing in their corresponding in vivo microenvironment than cultures grown on conventional substrata.

### Engineered Metastases Demonstrate Organ Specific Therapeutic Response

The identification of treatment regimens that are effective in treating metastases in a tissue specific manner remains an active area of interest as metastases in different organs of the same patient can respond differently to the same therapeutic regimen. To determine if the substrata upon which CRC cells are grown influences treatment responses, we treated CRC cells grown on plastic, collagen, Matrigel, liver BMSs and lung BMSs to standard CRC chemotherapy regimens as well as radiotherapy. Four commonly used chemotherapy treatment regimens for metastatic CRCs were examined: irinotecan alone, irinotecan+5-fluorouracil (5-FU), oxaliplatin alone, and oxaliplatin+5-FU. Applicants found that the responses of CRC cell lines to chemotherapy and radiotherapy were strongly influenced by their in vitro microenvironment (FIG. 5a). For example, engineered Caco2 lung metastases are uniformly more sensitive to chemotherapy regimens than engineered Caco2 liver metastases (FIG. 5a). Additionally, Applicants found that the responses of CRC cell cultures to radiotherapy were dependent upon their culture substrata. (FIG. 5b). Importantly, Applicants observed that the treatment responses of engineered liver and lung metastases differed. These results demonstrate that the treatment response of the CRC cells is impacted by the organ-specific composition of the extracellular matrix on which they are cultured.

The behavior of metastases is strongly influenced by the tissue specific microenvironments they inhabit. Recognizing the importance of this interaction, Applicants have developed a novel 3D in vitro culture platform using matrix extracts of decellularized organs to study metastatic disease in a tissue-specific manner. As proof-of-principle, Applicants engineered liver and lung metastases from CRC cell lines. While some studies use co-cultures to recreate the tumor microenvironment, this data demonstrates that Applicants are able to engineer metastases that possess histologic features and gene expression profiles that are similar to those present in metastatic lesions in vivo by recapitulating the acellular biochemical environment. For example, Applicants identified signet ring cells in the engineered liver metastases, an observation that has not been reported in any ex vivo cancer model system to date. Importantly, Applicants show that engineered metastases are more adapted to growth in their respective in vivo tissue specific microenvironments when compared to cells grown on conventional culture substrata. Applicants also observed that engineered liver metastases were capable of forming metastases in lung. Potential explanations for this observation are either the lung is a generally more “permissive” environment than the liver, or that tumor cells grown in a liver microenvironment develop characteristics that promote their ability to grow within the lung.

This data demonstrates that the therapeutic responses of CRC cells to standard treatment regimens are dependent upon the tissue-specific microenvironment to which they are exposed to in vitro. It is interesting that cells grown on lung BMSs are generally more sensitive to treatment than cells grown on liver BMSs. This data is consistent with the clinical observations that liver metastasis is the main cause of morbidity and mortality in metastatic CRC patients, even though both liver and lung metastases are common in this disease setting.

Not to be bound by theory, Applicants believe this culture platform, capable of engineering metastases in vitro, represents a powerful tool for the study of metastatic cancer biology in a tissue specific manner. Future work will involve identifying specific ECM components that affect cancer cell behavior as such evaluations may reveal novel therapeutic targets. This model is also useful for the study of tissue-specific treatment responses of metastases. Notably, this model can be utilized for drug screening assays to test newly developed therapeutics for metastatic disease. Importantly, this is the only system that allows for high throughput screening assays aimed at identifying therapeutics designed to treat metastases in an organ-specific manner.

The interaction between cancer cells and organ-specific environment is thought to play an important role in modulating the ability of cancer cells to colonize organs and subsequently grow into metastases, the “seed and soil” hypothesis². Recognizing the importance of the microenvironment, the “soil”, the tissue-specific biomatrix scaffolds were used and closely mimicked in vivo metastases behaviorally, histologically, and biologically. Additionally, the responses of engineered metastases to therapeutic treatments was dependent upon the organ-specific matrix chemistry on which the tumor cells were grown. Importantly, culturing the cells on biomatrix scaffolds increased the metastatic potential of the cancer cells and may have imparted tissue-specific predilections for tissue colonization. The findings implicate the potential to develop novel prognostics and therapeutic strategies for metastatic cancer patients and is unique given that it is the only culture system currently available that recapitulates organ-specific microenvironments ex vivo.

### REFERENCES

1 Fidler, I. J. The organ microenvironment and cancer metastasis. Differentiation; Research in Biological Diversity 70, 498-505, doi: doi:10.1046/j.1432-0436.2002.700904.x (2002).
2 Katt, M. E., Placone, A. L., Wong, A. D., Xu, Z. S. & Searson, P. C. In Vitro Tumor Models: Advantages, Disadvantages, Variables, and Selecting the Right Platform. (Review). Frontiers in Bioengineering and Biotechnology 4, 12, doi:doi:10.3389/fbioe.2016.00012 (2016).
3 Higashiyama, M. et al. Differences in chemosensitivity between primary and paired metastatic lung cancer tissues: In vitro analysis based on the collagen gel droplet embedded culture drug test (CD-DST). Journal of Thoracic Disease 4, 30-47, doi:doi:10.3978/j.issn.2072-1439.2011.05.02 (2012).
4 Bandyopadhyay, A. et al. Inhibition of pulmonary and skeletal metastasis by a transforming growth factor-beta type I receptor kinase inhibitor. Cancer research 66, 6714-6721, doi:10.1158/0008-5472.CAN-05-3565 (2006).
5 Higashiyama, M. et al. Differences in chemosensitivity between primary and paired metastatic lung cancer tissues: In vitro analysis based on the collagen gel droplet embedded culture drug test (CD-DST). J Thorac Dis 4, 30-47, doi:10.3978/j.issn.2072-1439.2011.05.02 (2012).
6 Badylak, S. F., Taylor, D. & Uygun, K. Whole-organ tissue engineering: decellularization and recellularization of three-dimensional matrix scaffolds. Review. Annual Review of Biomedical Engineering 13, 27-53, doi:doi:10.1146/annurev-bioeng-071910-124743 (2011).
7 Ott, H. C. et al. Perfusion-decellularize matrix using nature's platform to engineer a bioartificial heart. Nature Medicine 14, 213-221 (2008).
8 Baptista, P. M. et al. The use of whole organ decellularization for the generation of a vascularized liver organoid. Hepatology 53, 604-617 (2011).
9 Uygun, B. et al. Organ re-engineering through develpment of a transplantatble recellularized liver graft using decellularized liver matrix. Nature Medicine 16, 814-820 (2010).
10 Soto-Gutierrez, A. et al. Engineering of an hepatic organoid to develop liver assist devices. Cell Transplantation 19, 815-822, doi:doi: 10.3727/096368910X508933. (2010).
11 Petersen, T. H. et al. Tissue-engineered lungs for in vivo implantation. Science 329 538-541, doi:doi:10.1126/science.1189345. (2010).
12 Wang, Y. et al. Lineage restriction of hepatic stem cells to mature fates is made efficient by tissue-specific biomatrix scaffolds. Hepatology 53, 293-305 (2011).
13 Crapo, P. M., Gilbert, T. W. & Badylak, S. F. An overview of tissue and whole organ decellularization processes. Biomaterials 32, 3233-3243, doi:doi:10.1016/j.biomaterials.2011.01.057 (2011).
14 Flatmark, K., Maelandsmo, G. M., Martinsen, M., Rasmussen, H. & Fodstad, O. Twelve colorectal cancer cell lines exhibit highly variable growth and metastatic capacities in an orthotopic model in nude mice. European Journal of Cancer 40, 1593-1598, doi:doi:10.1016/j.ejca.2004.02.023 (2004).
15 Vinci, M. et al. Advances in establishment and analysis of three-dimensional tumor spheroid-based functional assays for target validation and drug evaluation. BMC Biology 10, 29, doi:doi: 10.1186/1741-7007-10-29. (2012).
16 Schelter, F. et al. Tumor cell-derived Timp-1 is necessary for maintaining metastasis-promoting Met-signaling via inhibition of Adam-10. Clinical & experimental metastasis 28, 793-802, doi:10.1007/s10585-011-9410-z (2011).
17 Weidle, U. H., Birzele, F. & Kruger, A. Molecular targets and pathways involved in liver metastasis of colorectal cancer. Clinical & experimental metastasis 32, 623-635, doi:10.1007/s10585-015-9732-3 (2015).
18 Chambers, A. F., Groom, A. C. & MacDonald, I. C. Dissemination and growth of cancer cells in metastatic sites. Nature Reviews Cancer 2, 563-572, doi:10.1038/nrc865 (2002).
19 Ring, B. Z. & Ross, D. T. Predicting the sites of metastases. Genome Biol 6, doi:Artn 24110.1186/Gb-2005-6-12-241 (2005).
20 Fidler, I. J. The pathogenesis of cancer metastasis: the ‘seed and soil’ hypothesis revisited. Nature Reviews—Cancer 3, 1-7 (2002).
21 Katt, M. E., Placone, A. L., Wong, A. D., Xu, Z. S. & Searson, P. C. In Vitro Tumor Models: Advantages, Disadvantages, Variables, and Selecting the Right Platform. Frontiers in bioengineering and biotechnology 4, 12, doi:10.3389/fbioe.2016.00012 (2016).
22 Klaas, M. et al. The alterations in the extracellular matrix composition guide the repair of damaged liver tissue. Sci Rep—Uk 6, doi:Artn 27398 10.1038/Srep27398 (2016).

1. A method of determining a potential of a cancer or tumor to metastasize within a patient diagnosed with a cancer or tumor, comprising:
seeding one or more biomatrix scaffolds with cells of a tumor biopsy or sample taken from a patient diagnosed with a cancer or tumor, each one or more biomatrix scaffolds originating from one or more predetermined tissues or organs, respectively, of a human body
analyzing the cells for the ability to form a colony of growing cells,
wherein each biomatrix scaffold recapitulates the biological facets of the organ or tissue from which it originated, and
wherein in vivo metastasis into the one or more predetermined tissues or organs is predicted if the cells form a colony of growing cells on one or more biomatrix scaffolds, respectively, originating therefrom.

2. The method of claim 1 in which the metastasis into the one or more predetermined tissues or organs is predicted if the cells form a colony of growing cells on the substratum of one or more biomatrix scaffolds, respectively, originating therefrom.

3. The method of claim 1 in which each of the one or more biomatrix scaffolds originating from one or more predetermined tissues or organs is selected from a frozen section of biomatrix scaffold, a cryopulverized biomatrix scaffold, and an intact biomatrix scaffold.

4. The method of claim 1 further comprising characterizing the colony based on its histology, exosome production, microRNA production, interactions with mesenchymal cells, and/or gene expression profile.

5. The method of claim 1 further comprising screening the colony for responsiveness to one or more cancer therapies and/or chemotherapeutic agents.

6. The method of claim 5 wherein the cancer therapies and/or chemotherapeutic agents are selected based on histology exosome production, microRNA production, interactions with mesenchymal cells, and/or gene expression profile of the colony.

7. The method of claim 5 in which the one or more cancer therapies and/or chemotherapeutic agents are selected from one or more doses of radiation therapy and/or one or more chemotherapies such as an anthracycline, an alkylating agent, a platinum-based agent, an anti-metabolite, a topoisomerase inhibitor, and a mitotic inhibitor.

8. The method of claim 5 in which the one or more cancer therapies and/or chemotherapeutic agents are selected from radiation therapy, immunotherapy, endocrine therapy, and molecular therapy.

9. A method of determining the appropriate treatment for a cancer or tumor in a patient diagnosed with a cancer or tumor, comprising:
seeding one or more biomatrix scaffolds with (1) cells of a tumor biopsy or sample taken from the patient diagnosed with a cancer or tumor or (2) cells from a cell line of the same cancer or tumor type as that of the patient, each one or more biomatrix scaffolds originating from one or more predetermined tissues or organs, respectively, of a human body, wherein the cells to form a colony of growing cells on the biomatrix scaffold,
treating the colony to one or more cancer therapies and/or chemotherapeutic agents,
wherein the biomatrix scaffold recapitulates the biological facets of the tissue or organ from which it originated,
and wherein an appropriate treatment for the patient is selected based on the responsiveness of the colony to treatment with the one or more cancer therapies and/or chemotherapeutic agents.

10. The method of claim 9 wherein the cancer therapies and/or chemotherapeutic agents are selected based on histology or gene expression profile of the three-dimensional colony.

11. The method of claim 9 in which the one or more cancer therapies and/or chemotherapeutic agents are selected from one or more doses of radiation therapy and/or one or more chemotherapies such as an anthracycline, an alkylating agent, a platinum-based agent, an anti-metabolite, a topoisomerase inhibitor, and a mitotic inhibitor.

12. The method of claim 9 in which the one or more cancer therapies and/or chemotherapeutic agents are selected from radiation therapy, immunotherapy, endocrine therapy, and molecular therapy.

13. The method of claim 12 in which the immunotherapy is selected from dendritic cell therapy, antibody-dependent therapy, T-cell dependent therapy, or NK-cell dependent therapy.

14. The method of claim 12 in which the molecular therapy comprises administering one or more antisense oligonucleotides.

15. A kit comprising one or more biomatrix scaffolds originating from one or more predetermined tissues or organs, respectively, of a human body and instructions for seeding cells on the one or more biomatrix scaffolds and instructions for performing the method of claim 1.

16. A kit comprising one or more biomatrix scaffolds originating from one or more predetermined tissues or organs, respectively, of a human body and instructions for seeding cells on the one or more biomatrix scaffolds and instructions for performing the method of claim 9.

17. The kit of claim 16 further comprising one or more cancer therapies and/or chemotherapeutic agents.

18. An artificial tumor or metastases model specific to one or more predetermined tissues or organs of a human body generated by:
seeding one or more biomatrix scaffolds with (1) cells of a tumor biopsy or sample taken from the patient diagnosed with a cancer or tumor or (2) cells from a cell line of the same cancer or tumor type as that of the patient, each one or more biomatrix scaffolds originating from one or more predetermined tissues or organs, respectively, of a human body,
wherein the cells to form a colony of growing cells on the biomatrix scaffold, and
wherein the biomatrix scaffold recapitulates the biological facets of the tissue or organ from which it originated.
